(19) United States
(12) Patent Application Publication
(10) Pub. No.: US 2007/0128137 A1
(43) Pub. Date: Jun. 7, 2007

(54) Water in oil emulsion compositions containing siloxane elastomers

(76) Inventors: Naohisa Yoshimi, Nishinomiya (JP); Hidekazu Tanaka, Toyonaka (JP); Akihiro Ueda, Kobe (JP); Kosaku Yamada, Kobe (JP); Kara JoAnn Stump, Stonewood, WV (US); Chu Zhu, Cincinnati, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/607,220

(22) Filed: Dec. 1, 2006

Related U.S. Application Data
(60) Provisional application No. 60/742,073, filed on Dec. 2, 2005.
(60) Provisional application No. 60/800,554, filed on May 15, 2006.
(60) Provisional application No. 60/812,791, filed on Jun. 12, 2006.

(51) Int. Cl. A61K 8/89

(52) U.S. Cl. 424/62; 424/70.12

(57) ABSTRACT

The present invention relates to water in oil emulsion compositions comprising: from about 0.1% to about 15% of a non-emulsifying crosslinked siloxane elastomer; from about 0.1% to about 15% of an emulsifying crosslinked siloxane elastomer; from about 1% to about 40% of a solvent for the non-emulsifying and emulsifying crosslinked siloxane elastomers; optionally, from 0% to about 5% of an additional emulsifier; and from about 50% to about 99% of aqueous phase; wherein when shear stress is applied to the composition during spreading on skin, aqueous phase is released from the emulsion.

### CROSS REFERENCE TO RELATED APPLICATIONS

This application claims the benefit of U.S. Provisional Application Nos. 60/742073, filed Dec. 2, 2005; 60/800554, filed May 15, 2006; and 60/812791, filed Jun. 12, 2006. These applications are incorporated by reference.

### FIELD OF THE INVENTION

The present invention relates to water in oil emulsion type skin care compositions containing a combination of emulsifying and non-emulsifying siloxane elastomers. Such compositions are useful for delivering skin care actives in products with consumer acceptable aesthetics. In particular, the amount and rate of aqueous phase released from the compositions upon spreading application to the skin can provide consumer acceptable aesthetics and non-greasy skin feel.

### BACKGROUND

Many personal care products currently available to consumers are directed primarily to improving the health and/or physical appearance of the skin and/or hair. Among the skin care products, many are directed to hydration, whitening, oil control, delaying, minimizing or even eliminating skin wrinkling and other histological changes typically associated with the aging of skin or environmental damage to human skin. Numerous compounds have been described in the art as being useful for regulating skin conditions such as those listed above.

Skin is subject to insults by many extrinsic and intrinsic factors. Extrinsic factors include ultraviolet radiation (e.g., from sun exposure), environmental pollution, wind, heat, low humidity, harsh surfactants, abrasives, and the like. Intrinsic factors include chronological aging and other biochemical changes from within the skin. While delivery of specific skin care actives or compounds that can help condition the skin and/or alleviate the damage caused by such insults is of course important, consumer acceptance of the sensory and aesthetic aspects of a particular skin care composition is also important. For example, as the level of commonly incorporated moisturizing agents such as glycerin increases, greasy skin feel also increased. Many consumers dislike heavy, oily or greasy feeling compositions and prefer compositions that can provide smooth spreadability and water-like, fresh skin feel, with silky after-feel.

Previous skin care and cosmetic compositions incorporating silicone elastomers have been described, e.g., WO 02/03930, WO 02/03950; WO 02/03951; WO 02/03952; EP 1 166 746 A1; EP 1 068 851 A1; Japanese Laid Open Publication No. 2003-081757; and Japanese Laid Open Publication No. 2003-55141. Among these disclosures, compositions said to provide the impression of freshness and “splash” of the aqueous ingredients upon rubbing are described. However, such previous disclosures have shown compositions containing only silicone emulsifiers, or compositions using different silicone elastomer systems. Similarly, it is believed that previously disclosed compositions have not provided the level of aqueous content and release from the emulsion that the compositions of the present invention provide.

Based on the foregoing, there is a continuing need to formulate skin care compositions that can provide improved delivery of skin care actives while also providing sensory and aesthetic benefits, especially as related to non-greasy, fresh feeling and aqueous phase release. None of the existing art provides all of the advantages and benefits of the present invention.

### SUMMARY

The present invention relates to water in oil emulsion compositions comprising: from about 0.1% to about 15% of a non-emulsifying crosslinked siloxane elastomer; from about 0.1% to about 15% of an emulsifying crosslinked siloxane elastomer; from about 1% to about 40% of a solvent for the non-emulsifying and emulsifying crosslinked siloxane elastomers; optionally, from 0% to about 5% of an additional emulsifier; from about 50% to about 99% of aqueous phase; wherein when shear stress is applied to the composition during spreading on skin, aqueous phase is released from the emulsion.

The present invention also relates to methods of using such compositions to regulate the condition of mammalian skin. Said methods generally contain the step of topically applying a safe and effective amount of the composition to the skin of a mammal needing such treatment.

These and other features, aspects, and advantages of the present invention will become evident to those skilled in the art from a reading of the present disclosure.

### BRIEF DESCRIPTION OF THE DRAWINGS

FIGS. 1A-B are micrographs of suitable embodiments of the invention.

FIGS. 2A-C are micrographs of suitable embodiments of the invention.

FIGS. 3A-C are micrographs of a comparative example.

FIG. 4-6 are plots of log shear stress (x-axis) versus log viscosity (y-axis) for three suitable embodiments of the invention.

FIG. 7 is a plot of log shear stress (x-axis) versus log viscosity (y-axis) for a comparative example.

### DETAILED DESCRIPTION

While the specification concludes with the claims particularly pointing and distinctly claiming the invention, it is believed that the present invention will be better understood from the following description.

All percentages and ratios used herein are by weight of the total composition and all measurements made are at 25° C., unless otherwise designated.

As used herein, the “skin care products” are those used to treat or care for, or somehow moisturize, improve, or clean the skin. Products contemplated by the phrase “skin care products” include, but are not limited to moisturizers, personal cleansing products, occlusive drug delivery patches, nail polish, powders, wipes, hair conditioners, skin treatment emulsions, shaving creams and the like.

The term “ambient conditions” as used herein refers to surrounding conditions under about one atmosphere of pressure, at about 50% relative humidity, and at about 25° C., unless otherwise specified.

The compositions of the present invention can include, consist essentially of, or consist of, the components of the present invention as well as other ingredients described herein. As used herein, “consisting essentially of” means that the composition or component may include additional ingredients, but only if the additional ingredients do not materially alter the basic and novel characteristics of the claimed compositions or methods.

All percentages, parts and ratios are based upon the total weight of the skin care compositions of the present invention, unless otherwise specified. All such weights as they pertain to listed ingredients are based on the active level and, therefore, do not include carriers or by-products that may be included in commercially available materials, unless otherwise specified.

All publications cited herein are hereby incorporated by reference in their entirety.

The term “keratinous tissue,” as used herein, refers to keratin-containing layers disposed as the outermost protective covering of mammals (e.g., humans, dogs, cats, etc.) which includes, but is not limited to, skin, lips, hair, toenails, fingernails, cuticles, hooves, etc.

The term “dermatologically-acceptable,” as used herein, means that the compositions or components thereof so described are suitable for use in contact with mammalian keratinous tissue without undue toxicity, incompatibility, instability, allergic response, and the like.

The term “safe and effective amount” as used herein means an amount of a compound or composition sufficient to significantly induce a positive benefit, preferably a positive keratinous tissue appearance or feel benefit, or positive hair appearance or feel benefit, including independently or in combinations the benefits disclosed herein, but low enough to avoid serious side effects, i.e., to provide a reasonable benefit to risk ratio, within the scope of sound judgment of the skilled artisan.

The terms “smoothing” and “softening” as used herein mean altering the surface of the keratinous tissue such that its tactile feel is improved.

It is desirable to have one or more skin care actives at high levels for skin care benefits such as regulating the condition of skin. However, when high levels of skin care actives are used in traditional skin care products, there is a downside. For example, residue caused by “salting out” of niacinamide produces an undesirable whitening effect on the skin. Likewise, high levels of skin conditioning agents such as glycerin produces a greasy, sticky feeling on the skin.

Silicone elastomers are known in the art as useful components in skin care compositions. Such silicone elastomers have been used to reduce the tackiness/stickiness associated with skin conditioning agents including glycerin. The use of silicone emulsifiers, e.g, as described in EP 1068851 A1, has also been said to provide compositions that contain as much as 91% aqueous phase. However, it has been found that the water in oil emulsion compositions of the present invention comprising a mixture of emulsified and non-emulsified siloxane elastomers can provide skin care compositions that contain an aqueous phase which, upon release from the emulsion when applied by spreading onto the skin, provide even better sensory benefits than heretofor believed possible. In addition, the rate of aqueous phase release from the emulsions of the present invention can be controlled to provide the desired consumer aesthetic and sensory benefits. In addition, increased levels of skin conditioning agents such as glycerin can be incorporated into the compositions of the present invention, without causing the compositions to feel greasy or sticky when spread upon the skin.

The compositions of the present invention are also useful for regulating the condition of skin and especially for regulating keratinous tissue condition. Regulation of skin condition, namely mammalian and in particular human skin condition, is often required due to conditions which may be induced or caused by factors internal and/or external to the body. Examples include, environmental damage, radiation exposure (including ultraviolet radiation), chronological aging, menopausal status (e.g., post-menopausal changes in skin), stress, diseases, etc. For instance, “regulating skin condition” includes prophylactically regulating and/or therapeutically regulating skin condition, and may involve one or more of the following benefits: thickening of skin (i.e., building the epidermis and/or dermis and/or sub-dermal (e.g., subcutaneous fat or muscle) layers of the skin and where applicable the keratinous layers of the nail and hair shaft) to reduce skin atrophy, increasing the convolution of the dermal-epidermal border (also known as the rete ridges), preventing loss of skin elasticity (loss, damage and/or inactivation of functional skin elastin) such as elastosis, sagging, loss of skin recoil from deformation; non-melanin skin discoloration such as under eye circles, blotching (e.g., uneven red coloration due to, e.g., rosacea) (hereinafter referred to as “red blotchiness”), sallowness (pale color), discoloration caused by telangiectasia or spider vessels.

The compositions of the present invention provide additional benefits, including stability, absence of significant (consumer-unacceptable) skin irritation and good aesthetics.

The compositions of the present invention contain a non-emulsifying crosslinked siloxane elastomer; an emulsifying crosslinked siloxane elastomer; a solvent for the non-emulsifying and emulsifying crosslinked siloxane elastomers; optionally, an additional emulsifier; and aqueous water phase. The compositions also preferably contain one or more skin care actives.

The compositions herein may also include a wide variety of other ingredients. The compositions of the present invention are described in detail hereinafter.

Crosslinked Siloxane Elastomers

An essential component of the present invention is a crosslinked organopolysiloxane elastomer. No specific restriction exists as to the type of curable organopolysiloxane composition that can serve as starting material for the crosslinked organopolysiloxane elastomer. Examples in this respect are addition reaction-curing organopolysiloxane compositions which cure under platinum metal catalysis by the addition reaction between SiH-containing diorganopolysiloxane and organopolysiloxane having silicon-bonded vinyl groups; condensation-curing organopolysiloxane compositions which cure in the presence of an organotin compound by a dehydrogenation reaction between hydroxyl-terminated diorganopolysiloxane and SiH-containing diorganopolysiloxane; condensation-curing organopolysiloxane compositions which cure in the presence of an organotin compound or a titanate ester, by a condensation reaction between an hydroxyl-terminated diorganopolysiloxane and a hydrolyzable organosilane (this condensation reaction is exemplified by dehydration, alcohol-liberating, oxime-liberating, amine-liberating, amide-liberating, carboxyl-liberating, and ketone-liberating reactions); peroxide-curing organopolysiloxane compositions which thermally cure in the presence of an organoperoxide catalyst; and organopolysiloxane compositions which are cured by high-energy radiation, such as by gamma-rays, ultraviolet radiation, or electron beams.

Addition reaction-curing organopolysiloxane compositions are preferred for their rapid curing rates and excellent uniformity of curing. A particularly preferred addition reaction-curing organopolysiloxane composition is prepared from:

(A) an organopolysiloxane having at least 2 lower alkenyl groups in each molecule;

(B) an organopolysiloxane having at least 2 silicon-bonded hydrogen atoms in each molecule; and

(C) a platinum-type catalyst.

With regard to the above, component (A) is the basic component of the siloxane elastomer-generating organopolysiloxane, and curing proceeds by the addition reaction of this component with component (B) under catalysis by component (C). This component (A) must contain at least 2 silicon-bonded lower alkenyl groups in each molecule; an excellent cured product will not be obtained at few than two lower alkenyl groups because a network structure will not be formed. Said lower alkenyl groups are exemplified by vinyl, allyl, and propenyl. While the lower alkenyl groups can be present at any position in the molecule, their presence at the molecular terminals is preferred. The molecular structure of this component may be straight chain, branched straight chain, cyclic, or network, but a straight chain, possibly slightly branched, is preferred. The molecular weight of the component is not specifically restricted, and thus the viscosity may range from low viscosity liquids to very high viscosity gums. In order for the cured product to be obtained in the form of the rubbery elastomer, it is preferred that the viscosity at 25 degrees Centigrade be at least 100 centistokes. These organopolysiloxanes are exemplified by methylvinylsiloxanes, methylvinylsiloxane-dimethylsiloxane copolymers, dimethylvinylsiloxy-terminated dimethylpolysiloxanes, dimethylvinylsiloxy-terminated dimethylsiloxane-methylphenylsiloxane copolymers, dimethylvinylsiloxy-terminated dimethylsiloxane-diphenylsiloxane-methylvinylsiloxane copolymers, trimethylsiloxy-terminated dimethylsiloxane-methylvinylsiloxane copolymers, trimethylsiloxy-terminated dimethylsiloxane-methylphenylsiloxane-methylvinylsiloxane copolymers, dimethylvinylsiloxy-terminated methyl(3,3,3-trifluoropropyl) polysiloxanes, and dimethylvinylsiloxy-terminated dimethylsiloxane-methyl(3,3,-trifluoropropyl)siloxane copolymers.

Component (B) is an organopolysiloxane having at least 2 silicon-bonded hydrogen atoms in each molecule and is a crosslinker for component (A). Curing proceeds by the addition reaction of the silicon-bonded hydrogen atoms in this component with the lower alkenyl groups in component (A) under catalysis by component (C). This component (B) must contain at least 2 silicon-bonded hydrogen atoms in each molecule in order to function as a crosslinker. Furthermore, the sum of the number of alkenyl groups in each molecule of component (A) and the number of silicon-bonded hydrogen atoms in each molecule of component (B) is to be at least 5. Values below 5 should be avoided because a network structure is then essentially not formed.

No specific restriction exists on the molecular structure of this component, and it may be any of straight chain, branch-containing straight chain, cyclic, etc. The molecular weight of this component is not specifically restricted, but it is preferred that the viscosity at 25 degrees Centigrade be 1 to 50,000 centistokes in order to obtain good miscibility with component (A). It is preferred that this component be added in a quantity such that the molar ratio between the total quantity of silicon-bonded hydrogen atoms in the instant component and the total quantity of all lower alkenyl groups in component (A) falls within the range of (1.5:1) to (20:1). It is difficult to obtain good curing properties when this molar ratio falls below 0.5:1. When (20:1) is exceeded, there is a tendency for the hardness to increase to high levels when the cured product is heated. Furthermore, when an organosiloxane containing substantial alkenyl is supplementary added for the purpose of; for example, reinforcement, it is preferred that a supplemental addition of the instant SiH-containing component be made in a quantity offsetting these alkenyl groups. This component is concretely exemplified by trimethylsiloxy-terminated methylhydrogenpolysiloxanes, trimethylsiloxy-terminated dimethylsiloxane-methylhydrogensiloxane copolymers, and dimethylsiloxane-methylhydrogen-siloxane cyclic copolymers.

Component (C) is a catalyst of the addition reaction of silicon-bonded hydrogen atoms and alkenyl groups, and is concretely exemplified by chloroplatinic acid, possibly dissolved in an alcohol or ketone and this solution optionally aged, chloroplatinic acid-olefin complexes, chloroplatinic acid-alkenylsiloxane complexes, chloroplatinic acid-diketone complexes, platinum black, and carrier-supported platinum.

This component is added preferably at 0.1 to 1,000 weight parts, and more preferably at 1 to 100 weight parts, as platinum-type metal proper per 1,000,000 weight parts of the total quantity of components (A) plus (B). Other organic groups which may be bonded to silicon in the organopolysiloxane forming the basis for the above-described curable organopolysiloxane compositions are, for example, alkyl groups such as methyl, ethyl, propyl, butyl, and octyl; substituted alkyl groups such as 2-phenylethyl, 2-phenylpropyl, and 3,3,3-trifluoropropyl; aryl groups such as phenyl, tolyl, and xylyl; substituted aryl groups such as phenylethyl; and monovalent hydrocarbon groups substituted by, for example, the epoxy group, the carboxylate ester group, the mercapto group, etc.

Examples of the production of the organopolysiloxane elastomer powder are as follows: an organopolysiloxane composition as described above (additional-curable, condensation-curable, or peroxide-curable) is mixed with water in the presence of a surfactant (nonionic, anionic, cationic, or amphoteric), and, after mixing to homogeneity in a homomixer, colloid mill, homogenizer, propeller mixer, etc., this is cured by discharge into hot water (temperature at least 50 degrees Centigrade) and is then dried; the organopolysiloxane composition (addition-curable, condensation-curable, or peroxide-curable) is cured by spraying it directly into a heated current; the powder is obtained by curing a radiation-curable organopolysiloxane composition by spraying it under high energy radiation; the organopolysiloxane composition (addition-curable, condensation-curable, peroxide-curable) or high energy-curable organopolysiloxane composition is cured, the latter by high energy radiation, and the product is then pulverized using a known pulverizer such as, for example, a ball mill, atomizer, kneader, roll mill, etc., to thereby form the powder. Suitable organopolysiloxane elastomer powders include vinyl dimethicone/methicone silesquioxane crosspolymers like Shin-Etsu's KSP-100, KSP-101, KSP-102, KSP-103, KSP-104, KSP-105, hybrid silicone powders that contain a fluoroalkyl group like Shin-Etsu's KSP-200, and hybrid silicone powders that contain a phenyl group such as Shin-Etsu's KSP-300; and Dow Corning's DC 9506.

Preferred organopolysiloxane compositions are dimethicone/vinyl dimethicone crosspolymers. Such dimethicone/vinyl dimethicone crosspolymers are supplied by a variety of suppliers including Dow Coming (DC 9040 and DC 9041), General Electric (SFE 839), Shin Etsu (KSG-15, 16, 18 [dimethicone/phenyl vinyl dimethicone crosspolymer]), and Grant Industries (Gransil™ line of materials), and lauryl dimethicone/vinyl dimethicone crosspolymers supplied by Shin Etsu (e.g., KSG-21, KSG-210, KSG-310, KSG-320, KSG-41, KSG-42, KSG-43, KSG-44, KSG-710 and KSG-810). Cross-linked organopolysiloxane elastomers useful in the present invention and processes for making them are further described in U.S. Pat. No. 4,970,252 to Sakuta et al.; U.S. Pat. No. 5,760,116 to Kilgour et al.; U.S. Pat. No. 5,654,362 to Schulz, Jr. et al.; and Japanese Patent Application JP 61-18708, assigned to Pola Kasei Kogyo KK.

The compositions of the present invention comprise a combination of emulsifying and non-emulsifying crosslinked organopolysiloxane elastomer. The term “non-emulsifying,” as used herein, defines crosslinked organopolysiloxane elastomer from which polyoxyalkylene units or polyglycerin units are absent. The term “emulsifying,” as used herein, means crosslinked organopolysiloxane elastomer having at least one polyoxyalkylene (e.g., polyoxyethylene or polyoxypropylene) unit or polyglycerin unit.

Particularly useful emulsifying elastomers are polyoxyalkylene-modified elastomers formed from divinyl compounds, particularly siloxane polymers with at least two free vinyl groups, reacting with Si—H linkages on a polysiloxane backbone. Preferably, the elastomers are dimethyl polysiloxanes crosslinked by Si—H sites on a molecularly spherical MQ resin.

The non-emulsifying cross-linked organopolysiloxane elastomers of the present invention are preferably further processed by subjecting them to a high shear (approximately 5,000 psi) treatment in the presence of a solvent for the siloxane elastomer via a Sonolator with or without recycling in 10 to 60 passes.

The emulsifying crosslinked organopolysiloxane elastomer is present in the compositions of the present invention at concentrations of from about 0.1% to about 15%, preferably from about 0.2% to about 5%, most preferably from about 0.2% to about 2% by weight.

The non-emulsifying crosslinked organopolysiloxane elastomer is present in the compositions of the present invention at concentrations of from about 0.1 to about 15%, preferably from about 0.1 to about 5%, most preferably from about 0.1 to about 2% by weight.

Solvent for the Non-Emulsifying and Emulsifying Crosslinked Siloxane Elastomer

The compositions of the present invention comprise a solvent for the crosslinked organopolysiloxane elastomer described above. The solvent, when combined with the cross-linked organopolysiloxane elastomer particles of the present invention, serves to suspend and swell the elastomer particles to provide an elastic, gel-like network or matrix. The solvent for the cross-linked siloxane elastomer is liquid under ambient conditions, and preferably has a low viscosity to provide for improved spreading on the skin.

Concentrations of the solvent in the cosmetic compositions of the present invention will vary primarily with the type and amount of solvent and the cross-linked siloxane elastomer employed. Preferred concentrations of the solvent are from about 1% to about 50%, preferably from about 4% to about 50%, more preferably from about 5% to about 40%, by weight of the composition.

The solvent for the crosslinked siloxane elastomer comprises one or more liquid carriers suitable for topical application to human skin. These liquid carriers may be organic, silicone-containing or fluorine-containing, volatile or non-volatile, polar or non-polar, provided that the liquid carrier forms a solution or other homogenous liquid or liquid dispersion with the selected cross-linked siloxane elastomer at the selected siloxane elastomer concentration at a temperature of from about 28° C. to about 250° C., preferably from about 28° C. to about 100° C., preferably from about 28° C. to about 78° C. The solvent for the cross-linked siloxane elastomer preferably has a solubility parameter of from about 3 to about 13 (cal/cm³)^{0.5}, more preferably from about 5 to about 11 (cal/cm³)^{0.5}, most preferably from about 5 to about 9 (cal/cm³)^{0.5}. Solubility parameters for the liquid carriers or other materials, and means for determining such parameters, are well known in the chemical arts. A description of solubility parameters and means for determining them are described by C. D. Vaughan, “Solubility Effects in Product, Package, Penetration and Preservation” 103 Cosmetics and Toiletries 47-69, October 1988; and C. D. Vaughan, “Using Solubility Parameters in Cosmetics Formulation”, 36 J. Soc. Cosmetic Chemists 319-333, September/October, 1988, which articles are incorporated herein by reference.

The solvent preferably includes volatile, non-polar oils; non-volatile, relatively polar oils; non-volatile, non-polar oils; and non-volatile paraffinic hydrocarbon oils; each discussed more fully hereinafter. The term “non-volatile” as used herein refers to materials that exhibit a vapor pressure of no more than about 0.2 mm Hg at 25° C. at one atmosphere and/or to materials that have a boiling point at one atmosphere of at least about 300° C. The term “volatile” as used herein refers to all materials that are not “non-volatile” as previously defined herein. The phrase “relatively polar” as used herein means more polar than another material in terms of solubility parameter; i.e., the higher the solubility parameter the more polar the liquid. The term “non-polar” typically means that the material has a solubility parameter below about 6.5 (caucm³)^{0.5}.

1. Non-Polar, Volatile Oils

The non-polar, volatile oil tends to impart highly desirable aesthetic properties to the compositions of the present invention. Consequently, the non-polar, volatile oils are preferably utilized at a fairly high level. Non-polar, volatile oils particularly useful in the present invention are selected from the group consisting of silicone oils; hydrocarbons; and mixtures thereof. Such non-polar, volatile oils are disclosed, for example, in Cosmetics, Science, and Technology, Vol. 1, 27-104 edited by Balsam and Sagarin, 1972. The non-polar, volatile oils useful in the present invention may be either saturated or unsaturated, have an aliphatic character and be straight or branched chained or contain alicyclic or aromatic rings. Examples of preferred non-polar, volatile hydrocarbons include polydecanes such as isododecane and isodecane (e.g., Permethyl-99A which is available from Presperse Inc.) and the C7-C8 through C12-C15 isoparaffins (such as the Isopar Series available from Exxon Chemicals). Non-polar, volatile liquid silicone oils are disclosed in U.S. Pat. No. 4,781,917 issued to Luebbe et al. on Nov. 1, 1988. Additionally, a description of various volatile silicones materials is found in Todd et al., “Volatile Silicone Fluids for Cosmetics”, Cosmetics and Toiletries, 91:27-32 (1976). Particularly preferred volatile silicone oils are selected from the group consisting of cyclic volatile silicones corresponding to the formula: 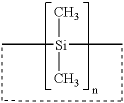
wherein n is from about 3 to about 7; and linear volatile silicones corresponding to the formula:
(CH₃)₃Si—O—[Si(CH₃)₂—O]ₘ—Si(CH₃)₃
wherein m is from about 1 to about 7. Linear volatile silicones generally have a viscosity of less than about 5 centistokes at 25° C., whereas the cyclic silicones have viscosities of less than about 10 centistokes at 25° C. Highly preferred examples of volatile silicone oils include cyclomethicones of varying viscosities, e.g., Dow Coming 200, Dow Coming 244, Dow Coming 245, Dow Coming 344, and Dow Coming 345, (commercially available from Dow Coming Corp.); SF-1204 and SF-1202 Silicone Fluids (commercially available from G.E. Silicones), GE 7207 and 7158 (commercially available from General Electric Co.); and SWS-03314 (commercially available from SWS Silicones Corp.).

2. Relatively Polar, Non-Volatile Oils

The non-volatile oil is “relatively polar” as compared to the non-polar, volatile oil discussed above. Therefore, the non-volatile co-solvent is more polar (i.e., has a higher solubility parameter) than at least one of the non-polar, volatile oils. Relatively polar, non-volatile oils potentially useful in the present invention are disclosed, for example, in Cosmetics, Science, and Technology, Vol. 1, 27-104 edited by Balsam and Sagarin, 1972; U.S. Pat. No. 4,202,879 issued to Shelton on May 13, 1980; and U.S. Pat. No. 4,816,261 issued to Luebbe et al. on Mar. 28, 1989. Relatively polar, non-volatile oils useful in the present invention are preferably selected from the group consisting of silicone oils; hydrocarbon oils; fatty alcohols; fatty acids; esters of mono and dibasic carboxylic acids with mono and polyhydric alcohols; polyoxyethylenes, polyoxypropylenes, mixtures of polyoxyethylene and polyoxypropylene ethers of fatty alcohols; and mixtures thereof. The relatively polar, non-volatile co-solvents useful in the present invention may be either saturated or unsaturated, have an aliphatic character and be straight or branched chained or contain alicyclic or aromatic rings. More preferably, the relatively polar, non-volatile liquid co-solvent are selected from the group consisting of fatty alcohols having from about 12-26 carbon atoms; fatty acids having from about 12-26 carbon atoms; esters of monobasic carboxylic acids and alcohols having from about 14-30 carbon atoms; esters of dibasic carboxylic acids and alcohols having from about 10-30 carbon atoms; esters of polyhydric alcohols and carboxylic acids having from about 5-26 carbon atoms; ethoxylated, propoxylated, and mixtures of ethoxylated and propoxylated ethers of fatty alcohols with from about 12-26 carbon atoms and a degree of ethoxylation and propoxylation of below about 50; and mixtures thereof. More preferred are propoxylated ethers of C14-C18 fatty alcohols having a degree of propoxylation below about 50, esters of C2-C8 alcohols and C12-C26 carboxylic acids (e.g. ethyl myristate, isopropyl palmitate), esters of C12-C26 alcohols and benzoic acid (e.g. Finsolv TN supplied by Finetex), diesters of C2-C8 alcohols and adipic, sebacic, and phthalic acids (e.g., diisopropyl sebacate, diisopropyl adipate, di-n-butyl phthalate), polyhydric alcohol esters of C6-C26 carboxylic acids (e.g., propylene glycol dicaprate/dicaprylate, propylene glycol isostearate); and mixtures thereof. Even more preferred are branched-chain aliphatic fatty alcohols having from about 12-26 carbon atoms.

3. Non-Polar, Non-Volatile Oils

In addition to the liquids discussed above, the solvent for the cross-linked siloxane elastomer may optionally include non-volatile, non-polar oils. Typical non-volatile, non-polar emollients are disclosed, for example, in Cosmetics, Science, and Technology, Vol. 1, 27-104 edited by Balsam and Sagarin, 1972; U.S. Pat. No. 4,202,879 issued to Shelton on May 13, 1980; and U.S. Pat. No. 4,816,261 issued to Luebbe et al. on Mar. 28, 1989. The non-volatile oils useful in the present invention are essentially non-volatile polysiloxanes, paraffinic hydrocarbon oils, and mixtures thereof. The polysiloxanes useful in the present invention selected from the group consisting of polyalkylsiloxanes, polyarylsiloxanes, polyalkylarylsiloxanes, poly-ethersiloxane copolymers, and mixtures thereof. Examples of these include polydimethyl siloxanes having viscosities of from about 1 to about 100,000 centistokes at 25° C. Among the preferred non-volatile silicone emollients useful in the present compositions are the polydimethyl siloxanes having viscosities from about 2 to about 400 centistokes at 25° C. Such polyalkylsiloxanes include the Viscasil series (sold by General Electric Company) and the Dow Corning 200 series (sold by Dow Coming Corp.). Polyalkylarylsiloxanes include polymethylphenyl siloxanes having viscosities of from about 15 to about 65 centistokes at 25° C. These are available, for example, as SF 1075 methyl-phenyl fluid (sold by General Electric Company) and 556 Cosmetic Grade Fluid (sold by Dow Coming Corp.).

Non-volatile paraffinic hydrocarbon oils useful in the present invention include mineral oils and certain branched-chain hydrocarbons. Examples of these fluids are disclosed in U.S. Pat. No. 5,019,375 issued to Tanner et al. on May 28, 1991. Preferred mineral oils have the following properties:

(1) viscosity from about 5 centistokes to about 70 centistokes at 40° C.;

(2) density between about 0.82 and 0.89 g/cm3 at 25° C.;

(3) flash point between about 138° C. and about 216° C.; and

(4) carbon chain length between about 14 and about 40 carbon atoms.

Preferred branched chain hydrocarbon oils have the following properties:

(1) density between about 0.79 and about 0.89 g/cm3 at 20° C.

(2) boiling point greater than about 250° C.; and

(3) flash point between about 110° C. and about 200° C.

Suitable branched-chain hydrocarbons include Permethyl 103 A, which contains an average of about 24 carbon atoms; Permethyl 104A, which contains an average of about 68 carbon atoms; Permethyl 102A, which contains an average of about 20 carbon atoms; all of which may be purchased from Permethyl Corporation; and Ethylflo 364 which contains a mixture of 30 carbon atoms and 40 carbon atoms and may be purchased from Ethyl Corp.

Additional solvents useful herein are described in U.S. Pat. No. 5,750,096 to Gerald J. Guskey et al., issued May 12, 1998.

Aqueous Phase

The cosmetic compositions of the present invention comprise an aqueous phase comprising from about 50% to about 99%, preferably from about 50% to about 95%, more preferably from about 65% to about 90% by weight of the composition.

The compositions of the present invention are water in oil emulsions. As such, generally speaking, there is weak bonding of aqueous phase to oil phase. This can permit the composition to transform upon application, e.g., to provide a water-splash sensation during spreading or rubbing upon the skin. For example, at the initial application to the skin but before spreading upon the skin, the composition is in the form of a gel or cream. Upon spreading, the finger shear stress is believed to break the emulsion, thereby releasing the aqueous phase from the emulsion. This provides good consumer sensory benefit, as the aqueous phase so released is perceptible to the touch as well as visually.

In certain embodiments, the visually perceptible release of the aqueous phase may be characterized by the Microscopy Method as presented in the Test Methods. The microscopy method is a microscope-assisted visual analysis of the presence and size of the aqueous domains emulsified within the oil phase. The emulsion is subjected to timed increments of shear after which a micrograph of the emulsion is taken. A visually perceptible release of the aqueous phase occurs when an amorphous aqueous region having a maximum linear dimension of at least about 10 microns becomes visible at 500× magnification within about 1 minute of shear. In alternate embodiments, the visually perceptible release of the aqueous phase occurs when an amorphous region of water having a size of at least about 25, 50, or 75 microns becomes visible at 500× magnification within about 1 minute of shear. In another suitable embodiment, the visually perceptible release of the aqueous phase occurs when an amorphous region of water having a size of at least 10 microns becomes visible at 500× magnification within about 45 second, 30 second, or 15 seconds of shear.

In certain embodiments, the visually perceptible release of the aqueous phase may be characterized by phase separation after milling according to the Milling Method provided in the Test Methods. The milling method involves the bulk milling of a 30 g sample of the emulsion. In one embodiment, a visually perceptible release of a portion of the aqueous phase occurs when at least about 0.5 g of the aqueous phase separates after 1 minute of milling at a rate of 24000 rpm. In further embodiments, at least about 1.0 g, 2.5 g, or 5.0 g of the aqueous phase separates after 1 minute of milling at a rate of 24000 rpm. In another embodiment, a visually perceptible release of a portion of the aqueous phase occurs when at least 0.25 g of the aqueous phase separates after 1 minute of milling at a rate of 13500 rpm. In other embodiments, the composition may result in the separation of at least about a 0.5 g portion of the aqueous phase after 1 minute of milling at a rate of 24000 rpm while yielding no visually perceptible release of the aqueous phase (i.e., <0.1 g of aqueous phase) after 1 minute of milling at a rate of 8000 rpm. Such an embodiment is believed to have suitable shelf and processing stability while still exhibiting a perceptible release of the aqueous phase during typical skin application.

In certain embodiments, the tactilely perceptible release of the aqueous phase may be characterized by a viscosity drop as measured in the Rheological Method provided in the Test Methods. The rheological method involves applying a controlled stress to a sample of the emulsion to generate a rheology profile of the log of viscosity (y-axis) versus the log of shear stress (x-axis). For an emulsion exhibiting an aqueous phase release upon application of shear, the plot of viscosity versus shear yields a sharp decrease in viscosity at a critical shear stress. The slope of the region of the plot exhibiting a sharp decrease is less than about −5. In alternate embodiments, slope of the region of the plot exhibiting a sharp decrease is less than about −10, −25, −50, −75, or −100.

Without being bound by theory, the amount of aqueous phase released from the emulsion and the rate at which the aqueous phase is released from the emulsion can be controlled, depending upon how the oil phase is bonded to the aqueous phase in the emulsion. In addition, it is also believed that the amount of aqueous phase released from the emulsion and the rate at which it is released from the emulsion can be controlled, for example, by incorporating an additional emulsifier for dispersing the aqueous phase as described below, by changing the level of the emulsifying crosslinked siloxane elastomer within the claimed range, and by varying the water/oil phase ratio.

Additional Emulsifier for Dispersing the Aqueous Phase

The water-in-oil emulsions of the present invention can optionally contain an emulsifier in addition to an emulsifying elastomer. In some embodiments, the composition may contain from about 0% to about 5%, preferably from 0.01% to about 5% additional emulsifier, more preferably from about 0.1% to about 3%, still more preferably from about 0.1% to about 2%, emulsifier by weight of the composition. The additional emulsifier if present helps disperse and suspend the aqueous phase within the continuous silicone phase.

A wide variety of emulsifying agents can be employed herein to form the preferred water-in-silicone emulsion. Known or conventional emulsifying agents can be used in the composition, provided that the selected emulsifying agent is chemically and physically compatible with components of the composition of the present invention, and provides the desired dispersion characteristics. Suitable emulsifiers include silicone emulsifiers, non-silicon-containing emulsifiers, and mixtures thereof, known by those skilled in the art for use in topical personal care products. Preferably these emulsifiers have an HLB value of or less than about 14, more preferably from about 2 to about 14, and still more preferably from about 4 to about 10. Emulsifiers having an HLB value outside of these ranges can be used in combination with other emulsifiers to achieve an effective weighted average HLB for the combination that falls within these ranges.

Silicone emulsifiers are preferred. A wide variety of silicone emulsifiers are useful herein. These silicone emulsifiers are typically organically modified organopolysiloxanes, also known to those skilled in the art as silicone surfactants. Useful silicone emulsifiers include dimethicone copolyols. These materials are polydimethyl siloxanes which have been modified to include polyether side chains such as polyethylene oxide chains, polypropylene oxide chains, mixtures of these chains, and polyether chains containing moieties derived from both ethylene oxide and propylene oxide. Other examples include alkyl-modified dimethicone copolyols, i.e., compounds which contain C2-C30 pendant side chains. Still other useful dimethicone copolyols include materials having various cationic, anionic, amphoteric, and zwitterionic pendant moieties.

Nonlimiting examples of dimethicone copolyols and other silicone surfactants useful as emulsifiers herein include polydimethylsiloxane polyether copolymers with pendant polyethylene oxide sidechains, polydimethylsiloxane polyether copolymers with pendant polypropylene oxide sidechains, polydimethylsiloxane polyether copolymers with pendant mixed polyethylene oxide and polypropylene oxide sidechains, polydimethylsiloxane polyether copolymers with pendant mixed poly(ethylene)(propylene)oxide sidechains, polydimethylsiloxane polyether copolymers with pendant organobetaine sidechains, polydimethylsiloxane polyether copolymers with pendant carboxylate sidechains, polydimethylsiloxane polyether copolymers with pendant quaternary ammonium sidechains; and also further modifications of the preceding copolymers containing pendant C2-C30 straight, branched, or cyclic alkyl moieties. Examples of commercially available dimethicone copolyols useful herein sold by Dow Coming Corporation are Dow Corning® 190, 193, Q2-5220, 2501 Wax, 2-5324 fluid, and 3225C (this later material being sold as a mixture with cyclomethicone). Cetyl dimethicone copolyol is commercially available under the trade name ABIL® EM-90, and also available as a mixture with polyglyceryl-4 isostearate (and) hexyl laurate and is sold under the tradename ABIL® WE-09 (available from Goldschmidt). Cetyl dimethicone copolyol is also commercially available as a mixture with hexyl laurate (and) polyglyceryl-3 oleate (and) cetyl dimethicone and is sold under the tradename ABIL® WS-08 (also available from Goldschmidt). Polydimethylsiloxyethyl dimethicone copolyol is commercially available under the trade names KF-6028, KF-6104, KF-6105, and KF-6106 (from Shin-Etsu). Other nonlimiting examples of dimethicone copolyols also include lauryl dimethicone copolyol, lauryl polydimethylsiloxyethyl dimethicone copolyol, dimethicone copolyol acetate, dimethicone copolyol adipate, dimethicone copolyolamine, dimethicone copolyol behenate, dimethicone copolyol butyl ether, dimethicone copolyol hydroxy stearate, dimethicone copolyol isostearate, dimethicone copolyol laurate, dimethicone copolyol methyl ether, dimethicone copolyol phosphate, and dimethicone copolyol stearate.

Dimethicone copolyol emulsifiers useful herein are described, for example, in U.S. Pat. No. 4,960,764, to Figueroa, Jr. et al.; European Patent No. EP 330,369, to SaNogueira; G. H. Dahms, et al., “New Formulation Possibilities Offered by Silicone Copolyols,” Cosmetics & Toiletries, vol. 110, pp. 91-100, March 1995; M. E. Carlotti et al., “Optimization of W/O-S Emulsions And Study Of The Quantitative Relationships Between Ester Structure And Emulsion Properties,” J. Dispersion Science And Technology, 13(3), 315-336 (1992); P. Hameyer, “Comparative Technological Investigations of Organic and Organosilicone Emulsifiers in Cosmetic Water-in-Oil Emulsion Preparations,” HAPPI 28(4), pp. 88-128 (1991); J. Smid-Korbar et al., “Efficiency and usability of silicone surfactants in emulsions,” Provisional Communication, International Journal of Cosmetic Science, 12, 135-139 (1990); and D. G. Krzysik et al., “A New Silicone Emulsifier For Water-in-Oil Systems,” Drug and Cosmetic Industry, vol. 146(4) pp. 28-81 (April 1990).

Among the non-silicone-containing emulsifiers useful herein are various non-ionic and anionic emulsifying agents such as sugar esters and polyesters, alkoxylated sugar esters and polyesters, C1-C30 fatty acid esters of C1-C30 fatty alcohols, alkoxylated derivatives of C1-C30 fatty acid esters of C1-C30 fatty alcohols, alkoxylated ethers of C1-C30 fatty alcohols, polyglyceryl esters of C1-C30 fatty acids, C1-C30 esters of polyols, C1-C30 ethers of polyols, alkyl phosphates, polyoxyalkylene fatty ether phosphates, fatty acid amides, acyl lactylates, soaps, and mixtures thereof.

Skin Care Active

The topical compositions of the present invention preferably also include at least one skin care active. Actives that are typically characterized as “water-soluble” as well as actives that are typically characterized as “oil-soluble” are suitable for formulation herein. Without being bound by theory, it is believed the present compositions provide versatility in formulating a variety of actives.

In any embodiment of the present invention, however, the actives useful herein can be categorized by the benefit they provide or by their postulated mode of action. However, it is to be understood that the actives useful herein can in some instances provide more than one benefit or operate via more than one mode of action. Therefore, classifications herein are made for the sake of convenience and are not intended to limit the active to that particular application or applications listed.

Non-limiting examples of skin care actives suitable herein include niacinamide, hexamidine compounds, whitening actives, peptides, sugar amines, and mixtures thereof.

Niacinamide

Niacinamide (or another solid at ambient temperature vitamin B₃ compound that is soluble in a solvent) is a preferred skin care active for use herein. The present invention preferably includes from about 2% to about 30%, more preferably from about 2% to about 20%, even more preferably from about 2% to about 10% of a vitamin B₃ compound.

As used herein, “niacinamide” means a compound having the formula: 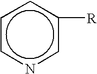
wherein R is —CONH₂.

The niacinamide may be included as the substantially pure material, or as an extract obtained by suitable physical and/or chemical isolation from natural (e.g., plant) sources. The vitamin B₃ compound is preferably substantially pure, more preferably essentially pure.

Hexamidine Compound

The topical compositions of the present invention comprise a safe and effective amount of one or more hexamidine and its salts. More preferably, the hexamidine is hexamidine diisethionate.

As used herein, “hexamidine” includes any isomers and tautomers of such and is commercially available as hexamidine diisethionate under the tradename Elestab® HP100 from Laboratoires Serobiologiques (Pulnoy, France).

In the composition of the present invention, the hexamidine preferably comprises from about 0.0001-25% by weight of the composition, more preferably from about 0.001% to about 10%, more preferably from about 0.01% to about 5%, and most preferably from about 0.02% to about 2.5%.

Whitening Agents

The present compositions may contain a whitening agent. The whitening agent useful herein refers to active ingredients that not only alter the appearance of the skin, but further improve hyperpigmentation as compared to pre-treatment. Useful whitening agents useful herein include ascorbic acid compounds, vitamin B₃ compounds, azelaic acid, butyl hydroxy anisole, gallic acid and its derivatives, hydroquinoine, kojic acid, arbutin, mulberry extract, undecylenoyl phenylalanine, and mixtures thereof. Use of combinations of whitening agents are also believed to be advantageous in that they may provide whitening benefit through different mechanisms.

When used, the compositions preferably contain from about 0.1% to about 10%, more preferably from about 0.2% to about 5%, by weight of the composition, of a whitening agent.

Ascorbic acid compounds are useful whitening agents, and have the formula (I): 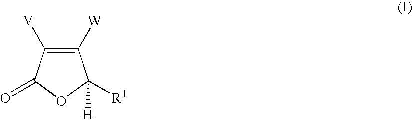
wherein V and W are independently —OH; R¹ is —CH(OH)—CH₂OH; and salts thereof.

Preferably, the ascorbic acid compound useful herein is an ascorbic acid salt or derivative thereof, such as the non-toxic alkali metal, alkaline earth metal and ammonium salts commonly known by those skilled in the art including, but not limited to, the sodium, potassium, lithium, calcium, magnesium, barium, ammonium and protamine salts which are prepared by methods well known in the art.

More preferably, the ascorbic acid salt useful herein is a metal ascorbate having the following formula (II): 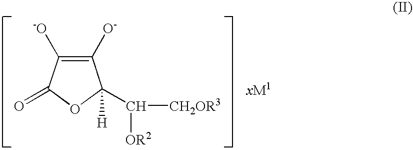
wherein R² and R³ are independently selected from hydrogen and linear or branched alkyl of 1 to about 8 carbons; M¹ is a metal; and x is an integer of from 1 to about 3. More preferably, R² and R³ are independently selected from hydrogen and linear or branched alkyl of 1 to about 3 carbons; M¹ is sodium, potassium, magnesium, or calcium.

Examples of other preferred ascorbic acid salts having formula (II) include monovalent metal salts (e.g., sodium ascorbate, potassium ascorbate), divalent metal salts (e.g., magnesium ascorbate, calcium ascorbate) and trivalent metal salts (e.g., aluminum ascorbate) of ascorbic acid.

Preferably, the ascorbic acid salt useful herein is a water soluble ascorbyl ester having the following formula (III): 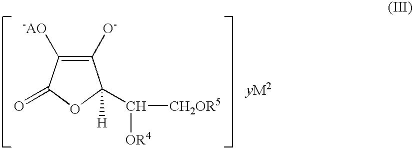
wherein A is sulfate or phosphate; R⁴ and R⁵ are independently selected from hydrogen and linear or branched alkyl of 1 to about 8 carbons; M² is a metal; and y is an integer of 1 to about 3. More preferably, R⁴ and R⁵ are independently selected from hydrogen and linear or branched 2 alkyl of 1 to about 3 carbons; M² is sodium, potassium, magnesium, or calcium.

Another particularly preferred ascorbic acid compound is 2-o-α-D-glucopyranosyl-L-ascorbic acid, usually referred to as L-ascorbic acid 2-glucoside or ascorbyl glucoside, and its metal salts. Such compounds are available from Hayashibara.

Magnesium ascorbyl phosphate is a stable form of vitamin C. In-vivo, it is converted to Vitamin C. It is soluble and stable in a variety of solvents including water, propylene glycol, 1,3-butylene glycol, maltitol, and glycerin. Unlike vitamin C, it is percutaneously absorbed into the skin. Magnesium ascorbyl phosphate is commercially available from Barnet Products Corp. as NIKKOL VC-PMG.

Exemplary water soluble salt derivatives include, but are not limited to, L-ascorbic acid 2-glucoside, L-ascorbyl phosphate ester salts such as sodium L-ascorbyl phosphate, potassium L-ascorbyl phosphate, magnesium L-ascorbyl phosphate, calcium L-ascorbyl phosphate, aluminum L-ascorbyl phosphate. L-ascorbyl sulfate ester salts can also be used. Examples are sodium L-ascorbyl sulfate, potassium L-ascorbyl sulfate, magnesium L-ascorbyl sulfate, calcium L-ascorbyl sulfate and aluminum L-ascorbyl sulfate.

Undecylenoyl Phenylalanine is the substituted amino acid that is also suitable for use herein as a whitening agent. It is available under the trade name Sepiwhite Msh, from Seppic. Cetyl Pyridinium Chloride and Tetrahydrocurcumin are also suitable for use herein as whitening agents.

Glycyrrhizic acid, a natural material derived from Glycyrrhiza Glabra, and its derivatives such as Glychrrhetinic Acid are also suitable for use herein. Such materials are available from Maurzen or Ichimaru Pharcos.

Peptides

Peptides, including but not limited to, di-, tri-, tetra-, and pentapeptides and derivatives thereof, may be included in the compositions of the present invention in amounts that are safe and effective. As used herein, “peptides” refers to both the naturally occurring peptides and synthesized peptides. Also useful herein are naturally occurring and commercially available compositions that contain peptides.

Suitable dipeptides for use herein include Carnosine (beta-ala-his). Suitable tripeptides for use herein include, gly-his-lys, arg-lys-arg, his-gly-gly. Preferred tripeptides and derivatives thereof include palmitoyl-gly-his-lys, which may be purchased as Biopeptide CL® (100 ppm of palmitoyl-gly-his-lys commercially available from Sederma, France); Peptide CK (arg-lys-arg); PEPTIDE CK+ (ac-arg-lys-arg-NH₂); and a copper derivative of his-gly-gly sold commercially as IAMIN, from Sigma (St. Louis, Mo.). Tetrapeptides and pentapeptides are also suitable for use herein.

A preferred commercially available pentapeptide derivative-containing composition is Matrixyl®, which contains 100 ppm of palmitoyl-lys-thr-thr-lys-ser (pal-KTTKS, commercially available from Sederma, France). Other preferred peptides include palmitoyl-lysine-threonine (pal-KT) and palmitoyl-glycine-glutamine-proline-arginine (pal-GQPR, available in a composition known as RIGIN®), also available from Sederma, France.

When included in the present compositions, peptides are preferably included in amounts of from about 1×10⁻⁶% to about 10%, more preferably from about 1×10⁻⁶% to about 0.1%, even more preferably from about 1×10⁻⁵% to about 0.01%, by weight of the composition. In certain compositions where the peptide is Carnosine®, the compositions preferably contain from about 0.1% to about 5%, by weight of the composition, of such peptides. In other embodiments wherein the peptide or peptide-containing composition palmitoyl-lys-thr-thr-lys-ser and/or Biopeptide CL® are included, the compositions preferably contain from about 0.0001% to about 10%, of palmitoyl-lys-thr-thr-lys-ser and/or Biopeptide CL® peptide-containing composition.

Sugar Amines

The compositions of the present invention may include a safe and effective amount of a sugar amine, which are also known as amino sugars. As used herein, “sugar amine” refers to an amine derivative of a six-carbon sugar. Preferably, the composition contains from about 0.001% to about 20%, more preferably from about 1% to about 10%, even more preferably from about 2% to about 5%, by weight of the composition, of the sugar amine.

Examples of sugar amines that are useful herein include glucosamine, N-acetyl glucosamine, mannosamine, N-acetyl mannosamine, galactosamine, N-acetyl galactosamine. Preferred for use herein is glucosamine. Additionally, combinations of two or more sugar amines may be used.

Skin Conditioning Agent

The topical compositions of the present invention include from about 1% to about 60%, by weight of the composition, of a skin conditioning agent. Preferably, the composition includes from about 2% to about 50%, more preferably from about 5% to about 40%, by weight of the composition, of the skin conditioning agent. Typically, compositions with a high percentage of a skin conditioning agent may be perceived as greasy or tacky by a user. It has been surprisingly found that compositions of the present invention may comprise a relatively high percentage (e.g., greater than 25%, by weight of the composition) of a skin conditioning agent without an appreciable increase in the perceived greasiness or tackiness.

Suitable skin conditioning agent for use herein include polyhydric alcohols such as polyalkylene glycols. Preferred for use herein are alkylene polyols and their derivatives. Examples of polyhydric alcohols useful herein include propylene glycol, dipropylene glycol, polypropylene glycol, polyethylene glycol, sorbitol, trehalose, hydroxypropyl sorbitol, hexylene glycol, 1,3-butylene glycol, 1,2,6-hexenetriol, glycerin, 1,2-hexanediol, pentylene glycol, ethoxylated glycerin, propoxylated glycerin, butanetriol, and mixtures thereof. A preferred polyhydric alcohol for use herein is glycerin.

The skin conditioning agent for use herein may be derived from any traditional means of manufacture and methods of purification.

Thickening Agents

The compositions of the present invention, in some embodiments, may further include one or more thickening agents. When present, the composition preferably includes from about 0.1% to about 5%, more preferably from about 0.1% to about 4%, and still more preferably from about 0.25% to about 3%, by weight of the composition of the thickening agent.

Nonlimiting classes of thickening agents include those selected from the following:

a) Carboxylic Acid Polymers

These polymers are crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol.

Examples of commercially available carboxylic acid polymers useful herein include the carbomers, which are homopolymers of acrylic acid crosslinked with allyl ethers of sucrose or pentaerytritol. The carbomers are available as the Carbopol® 900 series from B.F. Goodrich (e.g., Carbopol® 954). In addition, other suitable carboxylic acid polymeric agents include copolymers of C₁₀₋₃₀ alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol. These copolymers are known as acrylates/C10-30 alkyl acrylate crosspolymers and are commercially available as Carbopol® 1342, Carbopol® 1382, PEMULEN TR-1, and PEMULEN TR-2, from B.F. Goodrich. In other words, examples of carboxylic acid polymer thickeners useful herein are those selected from carbomers, acrylates/C₁₀-C₃₀ alkyl acrylate crosspolymers, and mixtures thereof.

b) Crosslinked Polyacrylate Polymers

The compositions of the present invention can optionally contain crosslinked polyacrylate polymers useful as thickeners or gelling agents including both cationic and nonionic polymers, with the cationics being generally preferred.

c) Polyacrylamide Polymers

The compositions of the present invention can optionally contain polyacrylamide polymers, especially nonionic polyacrylamide polymers including substituted branched or unbranched polymers. More preferred among these polyacrylamide polymers is the nonionic polymer given the CTFA designation polyacrylamide and isoparaffin and laureth-7, available under the Tradename Sepigel 305 from Seppic Corporation (Fairfield, N.J.).

Other polyacrylamide polymers useful herein include multi-block copolymers of acrylamides and substituted acrylamides with acrylic acids and substituted acrylic acids. Commercially available examples of these multi-block copolymers include HYPAN SR150H, SS500V, SS500W, SSSA100H, from Lipo Chemicals, Inc., (Patterson, N.J.).

d) Polysaccharides

A wide variety of polysaccharides are useful herein. “Polysaccharides” refer to gelling agents which contain a backbone of repeating sugar (i.e., carbohydrate) units. Nonlimiting examples of polysaccharide gelling agents include those selected from cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl substituted celluloses. In these polymers, the hydroxy groups of the cellulose polymer is hydroxyalkylated (preferably hydroxyethylated or hydroxypropylated) to form a hydroxyalkylated cellulose which is then further modified with a C₁₀-C₃₀ straight chain or branched chain alkyl group through an ether linkage. Typically these polymers are ethers of C₁₀-C₃₀ straight or branched chain alcohols with hydroxyalkylcelluloses. Examples of alkyl groups useful herein include those selected from stearyl, isostearyl, lauryl, myristyl, cetyl, isocetyl, cocoyl (i.e. alkyl groups derived from the alcohols of coconut oil), palmityl, oleyl, linoleyl, linolenyl, ricinoleyl, behenyl, and mixtures thereof. Preferred among the alkyl hydroxyalkyl cellulose ethers is the material given the CTFA designation cetyl hydroxyethylcellulose, which is the ether of cetyl alcohol and hydroxyethylcellulose. This material is sold under the tradename Natrosol® CS Plus from Aqualon Corporation (Wilmington, Del.).

Other useful polysaccharides include scleroglucans which are a linear chain of (1-3) linked glucose units with a (1-6) linked glucose every three units, a commercially available example of which is Clearogel™ CS11 from Michel Mercier Products Inc. (Mountainside, N.J.).

e) Gums

Other thickening and gelling agents useful herein include materials which are primarily derived from natural sources. Nonlimiting examples of these gelling agent gums include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof.

Particulate Material

The compositions of the present invention may, in some embodiments, contain a particulate material to modify skin feel or appearance. Examples of suitable particulate materials are disclosed in U.S. Pat. No. 5,997,887, to Ha, et al. Inorganic particulate materials, e.g., TiO₂, ZnO, or ZrO₂ are commercially available from a number of sources. One example of a suitable particulate material contains the material available from U.S. Cosmetics (TRONOX TiO₂ series, SAT-T CR837, a rutile TiO₂). Examples of particulate materials with inorganic chemical combination are, COVERLEAF AR-80 available in Catalysts and Chemicals Ind. Co. Ltd. which consists of layered inorganic chemicals, and Goddbal available in Suzuki Yushi Ind. Co. Ltd which is Silica powder encapsulating one or more inorganic chemicals. Preferably, particulate materials are present in the composition in levels of from about 0.01% to about 20%, more preferably from about 0.05% to about 15%, still more preferably from about 0.1% to about 12%, by weight of the composition.

Suitable organic powders/fillers include, but are not limited, to polymeric particles chosen from the methylsilsesquioxane resin microspheres such as for example those sold by Toshiba silicone under the name Tospearl 145A; microspheres of polymethylmethacrylates such as those sold by Seppic under the name Micropearl M 100; the spherical particles of crosslinked polydimethylsiloxanes, especially such as those sold by Dow Coming Toray Silicone under the name Trefil E 506C or Trefil E 505C, sphericle particles of polyamide and more specifically Nylon 12, especially such as those sold by Atochem under the name Orgasol 2002D Nat C05, polystyerene microspheres such as for example those sold by Dyno Particles under the name Dynospheres, ethylene acrylate copolymer sold by Kobo under the name FloBead EA209 and mixtures thereof.

The particulates may be hydrophobically treated to be more easily dispersed in the delivery vehicle. In addition, it may be useful to treat the pigments with a material that is compatible with a silicone phase. Particularly useful hydrophobic pigment treatments for use in water-in-silicone emulsions include polysiloxane treatments such as those disclosed in U.S. Pat. No. 5,143,722, incorporated herein by reference in its entirety. Also preferred are particulates having a primary average particle size of from about 10 nm to about 100,000 nm, more preferably from about 50 nm to about 5,000 nm, most preferably from about 100 nm to about 1000 nm. Mixtures of the same or different particulates having different particle sizes are also useful herein (e.g., incorporating a TiO2 having a primary particle size of from about 100 nm to about 400 nm with a TiO2 having a primary particle size of from about 10 nm to about 50 nm).

Optional Ingredients

The compositions of the present invention may contain one or more additional skin care actives. In a preferred embodiment, where the composition is to be in contact with human keratinous tissue, the additional components should be suitable for application to keratinous tissue, that is, when incorporated into the composition they are suitable for use in contact with human keratinous tissue without undue toxicity, incompatibility, instability, allergic response, and the like within the scope of sound medical judgment.

Non-limiting examples of additional skin care active ingredients that may be used in the present invention include sunscreen actives, oil-soluble terpene alcohols, phytosterols, oil-soluble vitamin compounds, additional vitamin B₃ compounds, oil-soluble vitamin compounds, emollients and occlusives, dehydroacetic acid, anti-acne actives, beta-hydroxy acids, chelators, flavonoids, anti-inflammatory agents, anti-cellulite agents, topical anesthetics, desquamation actives, anti-oxidants/radical scavengers, topical anesthetics, tanning actives, skin soothing and skin healing agents, anti-microbial and antifungal agents, and mixtures thereof.

The CTFA Cosmetic Ingredient Handbook, Eleventh Edition (2004) describes a wide variety of nonlimiting cosmetic and pharmaceutical ingredients commonly used in the skin care industry, which are suitable for use in the compositions of the present invention. Examples of these ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antioxidants, binders, biological additives, buffering agents, bulking agents, chelating agents, chemical additives, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, preservatives, propellants, reducing agents, sequestrants, skin bleaching and lightening agents (e.g., hydroquinone, kojic acid, ascorbic acid, ascorbyl glucosamine), skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents, thickeners, and vitamins and derivatives thereof.

In any embodiment of the present invention, however, the actives useful herein can be categorized by the benefit they provide or by their postulated mode of action. However, it is to be understood that the actives useful herein can in some instances provide more than one benefit or operate via more than one mode of action. Therefore, classifications herein are made for the sake of convenience and are not intended to limit the active to that particular application or applications listed.

Sunscreen Actives

Exposure to ultraviolet light can result in excessive scaling and texture changes of the stratum corneum. Therefore, the compositions of the subject invention may optionally contain a sunscreen active. As used herein, “sunscreen active” includes both sunscreen agents and physical sunblocks. Suitable sunscreen actives may be organic or inorganic.

Inorganic sunscreens useful herein include the following metallic oxides; titanium dioxide having an average primary particle size of from about 15 nm to about 100 nm, zinc oxide having an average primary particle size of from about 15 nm to about 150 nm, zirconium oxide having an average primary particle size of from about 15 nm to about 150 nm, iron oxide having an average primary particle size of from about 15 nm to about 500 nm, and mixtures thereof. When used herein, the inorganic sunscreens are present in the amount of from about 0.1% to about 20%, preferably from about 0.5% to about 10%, more preferably from about 1% to about 5%, by weight of the composition.

A wide variety of conventional organic sunscreen actives are suitable for use herein. Sagarin, et al., at Chapter VIII, pages 189 et seq., of Cosmetics Science and Technology (1972), discloses numerous suitable actives. Particulary preferred organic sunscreen actives useful in the compositions useful in the subject invention are homosalate, octocrylene, 2-ethylhexyl-p-methoxycinnamate (commercially available as PARSOL MCX), phenyl benzimidazole sulfonic acid, 2-hydroxy-4-methoxybenzophenone (Benzophenone-3), 2-ethylhexyl-salicylate, and mixtures thereof.

More preferred organic sunscreen actives useful in the compositions useful in the subject invention are 2-ethylhexyl-p-methoxycinnamate, butylmethoxydibenzoyl-methane, 2-hydroxy-4-methoxybenzo-phenone, 2-phenylbenzimidazole-5-sulfonic acid, octyldimethyl-p-aminobenzoic acid, octocrylene, zinc oxide, titanium dioxide, and mixtures thereof.

A safe and effective amount of the organic sunscreen active is used, typically from about 1% to about 20%, more typically from about 2% to about 10% by weight of the composition. Exact amounts will vary depending upon the sunscreen or sunscreens chosen and the desired Sun Protection Factor (SPF).

Oil-Soluble Terpene Alcohols

As used herein, “terpene alcohol” refers to organic compounds composed of two or more 5-carbon isoprene units [CH₂═C(CH₃)—CH═CH₂] with a terminal hydroxyl group.

Examples of oil-soluble terpene alcohols that are useful herein include farnesol, derivatives of farnesol, isomers of farnesol, geraniol, derivatives of geraniol, isomers of geraniol, phytantriol, derivatives of phytantriol, isomers of phytantriol, and mixtures thereof. Preferred for use herein is farnesol.

Farnesol is a naturally occurring substance which is believed to act as a precursor and/or intermediate in the biosynthesis of squalene and sterols, especially cholesterol. Farnesol is also involved in protein modification and regulation (e.g., farnesylation of proteins), and there is a cell nuclear receptor which is responsive to farnesol.

Chemically, farnesol is [2E,6E]-3,7,11-trimethyl-2,6,10-dodecatrien-1-ol and as used herein “farnesol” includes isomers and tautomers of such. Farnesol is commercially available, e.g., under the names farnesol (a mixture of isomers from Dragoco, 10 Gordon Drive, Totowa, N.J.) and trans-trans-farnesol (Sigma Chemical Company, P. O. Box 14508, St. Louis, Mo.). A suitable derivative of farnesol is farnesyl acetate which is commercially available from Aldrich Chemical Company, P. O. Box 2060, Milwaukee, Wis.

Geraniol is the common name for the chemical known as 3,7-dimethyl-2,6-octadien-1-ol. As used herein, “geraniol” includes isomers and tautomers of such. Geraniol is commercially available from Aldrich Chemical Company (P. O. Box 2060, Milwaukee, Wis.). Suitable derivatives of geraniol include geranyl acetate, geranylgeraniol, geranyl pyrophosphate, and geranylgeranyl pyrophosphate, all of which are commercially available from Sigma Chemical Company, P. O. Box 14508, St. Louis, Mo. For example, geraniol is useful as a spider vessel/red blotchiness repair agent, a dark circle/puffy eye repair agent, sallowness repair agent, a sagging repair agent, an anti-itch agent, a skin thickening agent, a pore reduction agent, oil/shine reduction agent, a post-inflammatory hyperpigmentation repair agent, wound treating agent, an anti-cellulite agent, and regulating skin texture, including wrinkles and fine lines.

Phytantriol is the common name for the chemical known as 3,7,11,15,tetramethylhexadecane-1,2,3,-triol. Phytantriol is commercially available from BASF (1609 Biddle Avenue, Whyandotte, Mich.). For example, phytantriol is useful as a spider vessel/red blotchiness repair agent, a dark circle/puffy eye repair agent, sallowness repair agent, a sagging repair agent, an anti-itch agent, a skin thickening agent, a pore reduction agent, oil/shine reduction agent, a post-inflammatory hyperpigmentation repair agent, wound treating agent, an anti-cellulite agent, and regulating skin texture, including wrinkles and fine lines.

Phytosterols

Phytosterol and derivatives thereof are known for providing skin lightening benefits. Non-limiting examples of oil-soluble phytosterol derivatives include β-sitosterol, campesterol, brassicasterol, lupenol, α-spinasterol, stigmasterol, their derivatives, and combinations thereof. More preferably, the phytosterol derivative is selected from the group consisting of β-sitosterol, campesterol, brassicasterol, stigmasterol, their derivatives, and combinations thereof.

Oil-Soluble Vitamin Compounds

A number of vitamins known by those in the art for providing various skin benefits are oil-soluble and some or all of their derivatives are oil-soluble. As such, these oil-soluble vitamin compounds are useful as oil-soluble skin care actives herein. Non-limiting examples of such oil-soluble vitamin compounds include retinoids, additional vitamin B₃ compounds, vitamin C (e.g. ascorbyl palmitate), vitamin D, vitamin K, vitamin E, and mixtures thereof. Preferred for use herein are retinoids, additional vitamin B₃ compounds, vitamin E, and mixtures thereof, each of which is discussed below.

As used herein, “retinoid” includes all natural and/or synthetic analogs of Vitamin A or retinol-like compounds which possess the biological activity of Vitamin A in the skin as well as the geometric isomers and stereoisomers of these compounds. Preferred retinoids are retinol, retinyl palmitate, retinyl acetate, retinyl propionate, retinal and combinations thereof, but any oil-soluble retinoid may be used herein.

The compositions of the present invention may also include, in some embodiments, an additional vitamin B₃ compound (other than niacinamide). When present, the composition preferably includes from about 0.01% to about 50%, more preferably from about 0.1% to about 10%, even more preferably from about 0.5% to about 10%, and still more preferably from about 1% to about 5%, by weight of the composition, of the vitamin B₃ compound.

As used herein, “additional vitamin B₃ compound” means a compound having the formula: 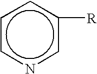
wherein R is, —COOH (i.e., nicotinic acid) or —CH₂OH (i.e., nicotinyl alcohol); derivatives thereof; and salts of any of the foregoing. Exemplary derivatives of the foregoing vitamin B₃ compounds include nicotinic acid esters, including non-vasodilating esters of nicotinic acid (e.g., tocopheryl nicotinate), nicotinyl amino acids, nicotinyl alcohol esters of carboxylic acids, nicotinic acid N-oxide and niacinamide N-oxide.

Vitamin E and several derivatives thereof are known to be especially useful as anti-oxidants/radical scavengers. Such antioxidants/radical scavengers are especially useful for providing protection against UV radiation which can cause increased scaling or texture changes in the stratum corneum and against other environmental agents which can cause skin damage.

Nonlimiting examples of oil soluble vitamin E compounds include tocopherol (vitamin E), tocopherol sorbate, tocopherol acetate, and other esters of tocopherol. Preferred anti-oxidants/radical scavengers are selected from tocopherol sorbate, tocopherol acetate, and mixtures thereof. Also useful herein are the class of materials, tocotrienols, which are related to vitamin E.

Emollients and Occlusives

Emollients are cosmetic ingredients which help to maintain the soft, smooth, and pliable appearance of skin. Emollients function by their ability to remain on the skin surface or in the stratum corneum to act as lubricants, to reduce flaking, and to improve the skin's appearance. Occlusives are cosmetic ingredients which retard with the evaporation of water from the skin surface. By blocking the evaporative loss of water, occlusive materials increase the water content of skin. Occlusive agents are generally lipids which tend to remain on the skin surface. Emollients may also sometimes exhibit occlusive properties upon application to the skin, and vice versa. Examples of suitable emollients and occlusives include Caprylic/Capric Glycerides, Isopropyl Isostearate, Mineral Oil, Cetyl Ricinoleate, and Petrolatum.

Dehydroacetic Acid

Dehydroacetic acid is a compound that is useful for regulating oily and/or shiny appearance of the skin, as disclosed in U.S. Pat. No. 6,150,403. Its chemical name is 3-Acetyl-6-methyl-2H-pyran-2,4(3H)-dione, and it can be commercially purchased from Universal Preserv-A-Chem of Brooklyn, N.Y. under the tradename Unisept DHA®, from Tri-K Industries (Northvale, N.J.), and under the tradename GEOGARD® 221 or GEOGARD® 361 from Lonza (Annandale, N.J.).

The compositions of the present invention may comprise from about 0.1% to about 10%, more preferably from about 0.5% to about 5%, and even more preferably from about 1% to about 5% of dehydroactic acid or dermatologically acceptable salts, derivatives, or tautomers thereof.

Hexanediol

The compositions of the present invention may comprise an effective amount of hexanediol, its isomers, tautomers, salts and derivatives. Some technical names for hexanediol suitable for use herein include 1,6-dihydroxyhexane, 1,6-hexanediol, hexamethylenediol, hexamethylene glycol, and 1,2-hexanediol.

The compositions of the present invention may comprise from about 0.0001% to about 50%, alternatively from about 0.001% to about 10%, alternatively from about 0.01% to about 5%, and alternatively from about 0.1% to about 2% hexanediol.

Anti-Acne Actives

The compositions of the present invention may contain a safe and effective amount of one or more anti-acne actives. Examples of useful anti-acne actives include resorcinol, sulfur, salicylic acid, benzoyl peroxide, erythromycin, zinc, etc.

Beta-Hydroxy Acids

Nonlimiting examples of oil-soluble beta-hydroxy acids include salicylic acid and derivatives thereof such as the octanoyl derivative. Beta-hydroxy acids are known to provide anti-acne and anti-aging benefits.

Chelators

As used herein, “chelator” or “chelating agent” means an active agent capable of removing a metal ion from a system by forming a complex so that the metal ion cannot readily participate in or catalyze chemical reactions. The inclusion of a chelating agent is especially useful for providing protection against UV radiation which can contribute to excessive scaling or skin texture changes and against other environmental agents which can cause skin damage. Preferred oil-soluble chelators useful in compositions of the subject invention are furildioxime, furilmonoxime, and derivatives thereof.

Flavonoids

Flavonoid compounds are broadly disclosed in U.S. Pat. Nos. 5,686,082 and 5,686,367. Nonlimiting examples of flavonoids useful herein include isoflavones, flavanones selected from the group consisting of unsubstituted flavanones, mono-substituted flavanones, and mixtures thereof; chalcones selected from the group consisting of unsubstituted chalcones, mono-substituted chalcones, di-substituted chalcones, tri-substituted chalcones, and mixtures thereof; flavones selected from the group consisting of unsubstituted flavones, mono-substituted flavones, di-substituted flavones, and mixtures thereof; one or more isoflavones; coumarins selected from the group consisting of unsubstituted coumarins, mono-substituted coumarins, di-substituted coumarins, and mixtures thereof; chromones selected from the group consisting of unsubstituted chromones, mono-substituted chromones, di-substituted chromones, and mixtures thereof; one or more dicoumarols; one or more chromanones; one or more chromanols; isomers (e.g., cis/trans isomers) thereof; and mixtures thereof. By the term “substituted” as used herein means flavonoids wherein one or more hydrogen atom of the flavonoid has been independently replaced with a hydroxyl, C1-C8 alkyl, or C1-C4 alkoxyl. Mixtures of the above flavonoid compounds may also be used.

Plant-derived isoflavones such as soy isoflavones are particularly useful herein. A particularly useful type of flavonoid herein is glycoside flavonoid, preferably selected from the group consisting of glucosyl hesperidin, glucosyl rutin, glucosyl myricitrin, glucosyl isoquercitrin, glucosyl quercitirin, methyl hesperedin, and mixtures thereof. Commercially available glycoside flavonoids include hesperidin, methyl hesperidin and rutin available from Alps Pharmaceutical Industry Co. Ltd. (Japan), and glucosyl hesperidin and glucosyl rutin available from Hayashibara Biochemical Laboratories, Inc. (Japan).

Anti-Inflammatory Agents

A safe and effective amount of an anti-inflammatory agent may be added to the compositions of the present invention, preferably from about 0.1% to about 10%, more preferably from about 0.5% to about 5%, of the composition. The anti-inflammatory agent enhances the skin appearance benefits of the present invention, e.g., such agents contribute to a more uniform and acceptable skin tone or color. The exact amount of anti-inflammatory agent to be used in the compositions will depend on the particular anti-inflammatory agent utilized since such agents vary widely in potency.

Steroidal anti-inflammatory agents, including but not limited to hydrocortisone, are suitable for use herein. Nonsteroidal anti-inflammatory agents, including but not limited to ibuprofen, naproxen, flufenamic acid, etofenamate, aspirin, mefenamic acid, meclofenamic acid, piroxicam and felbinac, are also suitable for use herein. The variety of compounds encompassed by these groups are well-known to those skilled in the art. Mixtures of non-steroidal anti-inflammatory agents may also be employed, as well as the dermatologically acceptable salts and esters of these agents.

“Natural” anti-inflammatory agents are also useful in the present invention. Such agents may suitably be obtained as an extract by suitable physical and/or chemical isolation from natural sources (e.g., plants, fungi, by-products of microorganisms) or can be synthetically prepared. For example, candelilla wax, bisabolol (e.g., alpha bisabolol), aloe vera, plant sterols (e.g., phytosterol), Manjistha (extracted from plants in the genus Rubia, particularly Rubia Cordifolia), and Guggal (extracted from plants in the genus Commiphora, particularly Commiphora Mukul), kola extract, chamomile, red clover extract, and sea whip extract, may be used.

Anti-Cellulite Agents

The compositions of the present invention may also contain a safe and effective amount of an anti-cellulite agent. Suitable agents may include, but are not limited to, xanthine compounds (e.g., caffeine, theophylline, theobromine, and aminophylline).

Topical Anesthetics

The compositions of the present invention may also contain a safe and effective amount of a topical anesthetic. Examples of topical anesthetic drugs include benzocaine, lidocaine, bupivacaine, chlorprocaine, dibucaine, etidocaine, mepivacaine, tetracaine, dyclonine, hexyl-caine, procaine, cocaine, ketamine, pramoxine, phenol, and pharmaceutically acceptable salts thereof.

Desquamation Actives

A safe and effective amount of a desquamation active may be added to the compositions of the present invention, preferably from about 0.1% to about 10%, more preferably from about 0.2% to about 5%, even more preferably from about 0.5% to about 4%, by weight of the composition. Desquamation actives enhance the skin appearance benefits of the present invention. For example, the desquamation actives tend to improve the texture of the skin (e.g., smoothness). One desquamation system that is suitable for use herein contains sulfhydryl compounds and zwitterionic surfactants and is described in U.S. Pat. No. 5,681,852, to Bissett, incorporated herein by reference. Another desquamation system that is suitable for use herein contains salicylic acid and zwitterionic surfactants and is described in U.S. Pat. No. 5,652,228 to Bissett, incorporated herein by reference. Zwitterionic surfactants such as described in these applications are also useful as desquamatory agents herein, with cetyl betaine being particularly preferred.

Anti-Oxidants/Radical Scavengers

The compositions of the present invention may include a safe and effective amount of an anti-oxidant/radical scavenger. The anti-oxidant/radical scavenger is especially useful for providing protection against UV radiation which can cause increased scaling or texture changes in the stratum corneum and against other environmental agents which can cause skin damage.

A safe and effective amount of an anti-oxidant/radical scavenger may be added to the compositions of the subject invention, preferably from about 0.1% to about 10%, more preferably from about 0.1% to about 5%, of the composition.

Anti-oxidants/radical scavengers such as ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, BHT, 6-hydroxy-2,5,7,8-tetramethylchroman-2-carboxylic acid (commercially available under the tradename Trolox®), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts may be used. Preferred anti-oxidants/radical scavengers are selected from tocopherol acetate and other esters of tocopherol, more preferably tocopherol acetate. The use of tocopherol sorbate in topical compositions and applicable to the present invention is described in U.S. Pat. No. 4,847,071, issued on Jul. 11, 1989 to Donald L. Bissett, Rodney D. Bush and Ranjit Chatterjee.

Tanning Actives

The compositions of the present invention may contain a tanning active. When present, it is preferable that the compositions contain from about 0.1% to about 20%, more preferably from about 2% to about 7%, and still more preferably from about 3% to about 6%, by weight of the composition, of dihydroxyacetone as an artificial tanning active.

Dihydroxyacetone, which is also known as DHA or 1,3-dihydroxy-2-propanone, is a white to off-white, crystalline powder. This material can be represented by the chemical formula C₃H₆O₃.

Skin Soothing and Skin Healing Actives

The compositions of the present invention may include a skin soothing or skin healing active. Skin soothing or skin healing actives suitable for use herein include panthenoic acid derivatives (including panthenol, dexpanthenol, ethyl panthenol), aloe vera, allantoin, bisabolol, and dipotassium glycyrrhizinate. A safe and effective amount of a skin soothing or skin healing active may be added to the present composition, preferably, from about 0.001% to about 30%, more preferably from about 0.01% to about 20%, still more preferably from about 0.01% to about 10%, by weight of the composition formed.

Antimicrobial and Antifungal Actives

The compositions of the present invention may contain an antimicrobial or antifungal active. Such actives are capable of destroying microbes, preventing the development of microbes or preventing the pathogenic action of microbes and are known to those of skill in the art. A safe and effective amount of an antimicrobial or antifungal active may be added to the present compositions, preferably, from about 0.001% to about 10%, more preferably from about 0.01% to about 5%, and still more preferably from about 0.05% to about 2%.

Solvents for Oil-Soluble Actives

If oil-soluble actives are used for purpose of sunscreen, whitening, anti-oxidant, etc, an ester may be used as a solvent to ensure efficacy of the oil-soluble active(s). A wide variety of suitable ester compounds are known and may be used herein and numerous examples can be found in “International Cosmetic Ingredient Dictionary and Handbook, 11th Edition, 2004”. Examples of suitable esters include esters of amino acids and C2-C8 alcohols such as Isopropyl Lauroyl Sarcosinate (Eldew SL205 from Ajinomoto), and esters of benzoic acid and C2-C8 alcohols such as Phenethyl Benzoate (X-tend 226 from International Specialty Products). The level of solvent to be used will depend on the type and amount oil-soluble active to be incorporated and can readily be determined by those of skill in the art.

Other Optional Ingredients

A variety of additional ingredients can be incorporated into the compositions of the present invention. Nonlimiting examples of these additional ingredients include; colorants, dyes, pigments; agents suitable for aesthetic purposes such as essential oils, fragrances, skin sensates, opacifiers, aromatic compounds (e.g., clove oil, menthol, camphor, eucalyptus oil, and eugenol); preservatives (e.g. alkyl esters of para-hydroxybenzoic acid, hydantoin derivatives such as 1,3-bis(hydroxymethyl)-5,5-dimthylhydantoin, propionate salts, and a variety of quaternary ammonium compounds such as benzalkonium chloride, quaternium 15 [Dowicil 200], benzethonium Chloride, and methylbenzethonium chloride). Particularly preferred preservatives are disodium EDTA, phenoxyethanol, ethyl paraben, methyl paraben, propyl paraben, imidazolidinyl urea (commercially available as Germall 1157), sodium dehydroacetate, benzyl alcohol and sodium benzoate.

Composition Preparation

The compositions useful for the methods of the present invention are generally prepared by conventional methods such as are known in the art of making topical compositions. Such methods typically involve mixing of the ingredients in one or more steps to a relatively uniform state, with or without heating, cooling, application of vacuum, and the like.

The topical compositions of the present invention may be formulated into a facial skin cosmetic, eye cosmetic, lip cosmetic, scalp hair styling aid, facial hair styling aid, moisturizer, wrinkle soothing serum, lotion, mascara, skin facial mask, skin lotion, skin cream, skin gel, eye gel, eye cream, lip gel, lip cream, cosmetic, foundation, or any other commonly known skin product or treatment.

Methods of Use

Applicants have found that the compositions of the present invention are useful in a variety of applications directed to enhancement of mammalian skin. The methods of use for the compositions disclosed and claimed herein include, but are not limited to: 1) methods of increasing the substantivity of a cosmetic to skin; 2) methods of moisturizing skin; 3) methods of improving the natural appearance of skin; 4) methods of applying a color cosmetic to skin; 5) methods of preventing, retarding, and/or treating wrinkles; 6) methods of providing UV protection to skin; 7) methods of preventing, retarding, and/or controlling the appearance of oil; 8) methods of modifying the feel and texture of skin; 9) methods of providing even skin tone; 10) methods of preventing, retarding, and/or treating the appear of spider vessels and varicose veins; 11) methods of masking the appearance of vellus hair on skin; and 12) methods of concealing blemishes and/or imperfections in human skin, including acne, age spots, freckles, moles, scars, under eye circles, birth marks, post-inflammatory hyperpigmentation, etc. Each of the methods discussed herein involve topical application of the claimed compositions to skin.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

Water in Oil emulsion skin care products are prepared by conventional methods from the following components.

| | Examples (values in wt %) | | | | | | | | | | | | |
|---|---|---|---|---|---|---|---|---|---|---|---|---|---|
| | 1 | 2 | 3 | 4 | 5 | 6 | 7 | 8 | 9 | 10 | 11 | 12 | 13 |
| Phase A | | | | | | | | | | | | | |
| Dimethicone *1 | 4.0 | 4.0 | 6.0 | 3.0 | 4.0 | 4.0 | 5.0 | 7.5 | 4.0 | 4.09 | 4.0 | 4.0 | 4.0 |
| Polymethyl | 4.0 | 4.0 | 6.0 | — | 4.0 | 4.0 | — | — | — | 4.09 | 4.0 | 4.0 | 4.0 |
| silsesquioxane *2 | | | | | | | | | | | | | |
| DC9040 *3 | 3.0 | 3.0 | 4.5 | — | — | 3.0 | — | — | — | 8.6 | 3.0 | 15.0 | 3.0 |
| DC9045 *4 | — | — | — | — | 3.0 | — | — | — | — | — | — | — | — |
| KSG-15 *5 | — | — | — | 2.5 | — | — | 2.7 | 2.7 | 2.7 | — | — | — | — |
| Cyclopenta- | 3.0 | 3.0 | 6.0 | — | 3.0 | 3.0 | 5.0 | 7.5 | 4.0 | 11.43 | 7.0 | 6.0 | 3.0 |
| siloxane *6 | | | | | | | | | | | | | |
| KSG-210 *7 | 2.5 | 5.0 | 4.0 | 5.0 | 2.75 | 2.75 | 2.3 | 2.3 | 2.3 | 5.37 | 2.75 | 2.75 | 2.75 |
| KF-6028 *8 | — | — | 0.15 | — | — | — | — | — | — | — | — | — | — |
| KF-6017 *9 | — | — | — | 0.3 | — | — | — | — | — | — | — | — | — |
| KF-6104 *10 | — | — | — | — | — | — | — | — | 0.5 | — | — | — | — |
| Cover Leaf AR-80 | — | — | 5.0 | — | — | — | — | — | — | — | — | — | — |
| 5% KF-9901 *11 | | | | | | | | | | | | | |
| KSG-18 *12 | — | — | — | 1.5 | — | — | — | — | — | — | — | — | — |
| DC-2503 *13 | — | — | — | — | — | — | — | — | — | 7.08 | — | 1.5 | — |
| Isopropyl | — | — | — | 2.2 | — | — | — | — | — | — | — | — | — |
| Isostearate | | | | | | | | | | | | | |
| Jeenate 3H *14 | — | — | — | — | — | — | — | — | — | 3.54 | — | — | — |
| TiO₂ Dispersion | — | — | — | — | — | — | — | — | — | 0.7 | — | — | — |
| *15 | | | | | | | | | | | | | |
| Petrolatum | — | — | — | — | — | — | — | — | — | — | — | 0.5 | — |
| Cetyl Ricinoleate | — | — | — | — | — | — | — | — | — | — | — | 0.5 | — |
| SEFA Cottonate | — | — | — | — | — | — | — | — | — | — | — | 0.5 | — |
| *16 | | | | | | | | | | | | | |
| Fragrance | 0.1 | 0.1 | 0.1 | — | — | — | — | — | — | 0.1 | — | 0.2 | 0.1 |
| Phase B | | | | | | | | | | | | | |
| Glycerin, USP | 10.0 | 10.0 | 30.0 | 5.0 | 7.0 | 10.0 | — | — | — | 10.0 | 10.0 | 10.0 | 10.0 |
| Niacinamide | 5.0 | 5.0 | 5.0 | 5.0 | 5.0 | 5.0 | 4.0 | 4.0 | 4.0 | 5.0 | 5.0 | 5.0 | 5.0 |
| Elestab HP100 | — | — | — | — | 0.1 | 0.1 | — | — | — | — | — | — | — |
| *17 | | | | | | | | | | | | | |
| Pentylene Glycol | 2.0 | 2.0 | 2.0 | 3.0 | — | 3.0 | 2.0 | 2.0 | 2.0 | 3.0 | | 3.0 | 3.0 |
| 1,2-Hexane Diol | — | — | — | — | 3.0 | — | — | — | — | — | 3.0 | — | — |
| Sodium Chloride | 0.5 | 0.5 | 0.5 | 0.5 | 0.5 | 0.5 | 0.5 | 0.5 | 0.5 | 0.5 | 0.5 | 0.5 | 0.5 |
| Panthenol | 0.5 | 0.5 | 0.5 | — | 1.0 | 1.0 | 0.5 | 0.5 | 0.5 | 0.5 | 0.5 | 0.5 | 0.5 |
| N-Acetyl | — | 2.0 | — | — | — | — | — | — | — | — | — | — | — |
| Glucosamine | | | | | | | | | | | | | |
| Promatrixyl ® | — | — | 0.353 | — | — | — | — | — | — | — | — | — | — |
| *18 | | | | | | | | | | | | | |
| Methylparaben | 0.2 | 0.2 | 0.2 | 0.2 | — | 0.2 | 0.2 | 0.2 | 0.2 | 0.2 | 0.2 | 0.2 | 0.2 |
| Sodium Citrate | 0.2 | 0.2 | 0.2 | 0.2 | — | 0.2 | 0.2 | 0.2 | 0.2 | 0.2 | 0.2 | 0.2 | 0.2 |
| Citric Acid | 0.03 | 0.03 | 0.03 | — | — | 0.03 | 0.015 | 0.015 | 0.015 | 0.03 | 0.03 | 0.03 | 0.03 |
| Sodium Benzoate | 0.07 | 0.07 | 0.07 | 0.07 | — | 0.07 | 0.07 | 0.07 | 0.07 | 0.07 | 0.05 | 0.05 | 0.07 |
| Ethylparaben | 0.05 | 0.05 | 0.05 | 0.05 | — | 0.05 | 0.05 | 0.05 | 0.05 | 0.05 | 0.05 | 0.05 | 0.05 |
| Benzyl Alcohol | — | — | — | 0.2 | — | — | — | — | — | — | — | — | — |
| Glydant Plus *19 | — | — | — | — | 0.3 | — | — | — | — | — | — | — | — |
| Disodium EDTA | — | — | — | 0.1 | — | 0.1 | 0.1 | 0.1 | 0.1 | — | — | — | — |
| Ascorbyl | — | — | — | 2.0 | — | — | — | — | — | — | — | — | — |
| Glucoside | | | | | | | | | | | | | |
| L-Arginine | — | — | — | 1.02 | — | — | — | — | — | — | — | — | — |
| Hexamidine | — | — | — | — | — | — | — | — | — | 0.1 | 0.1 | 0.1 | 0.1 |
| Diisethanoate | | | | | | | | | | | | | |
| Water | q.s. | q.s. | q.s. | q.s. | q.s. | q.s. | q.s. | q.s. | q.s. | q.s. | q.s. | q.s. | q.s. |
| | to | to | to | to | to | to | to | to | to | to | to | to | to |
| | 100 | 100 | 100 | 100 | 100 | 100 | 100 | 100 | 100 | 100 | 100 | 100 | 100 |

1. E.g., KF96A (6cs). Available from Shin-Etsu, Tokyo, Japan.
2. E.g., Tospearl 145A, CF 600, or 2000. Available from GE Advanced Materials, Wilton, CT.
3. 12.5% Dimethicone Crosspolymer in Cyclopentasiloxane. Available from Dow Corning, Midland, MI.
4. 12.5% Dimethicone in Cyclopentasiloxane. Available from Dow Corning, Midland, MI.
5. 5% Dimethicone/Vinyl Dimethicone Crosspolymer in Dimethicone. Available from Shin-Etsu, Tokyo, Japan.
6. E.g., SF-1202 available from GE Advanced Materials, Wilton, CT; SH245 available from Dow Corning, Midland, MI.
7. 25% Dimethicone PEG-10/15 Crosspolymer in Dimethicone. Available from Shin-Etsu, Tokyo, Japan.
8. PEG-9 Polydimethylsiloxyethyl Dimethicone. Available from Shin-Etsu, Tokyo, Japan.
9. PEG-10 Dimethicone. Available from Shin-Etsu, Tokyo, Japan.
10. Polyglyceryl-3 Polydimethylsiloxyethyl Dimethicone. Available from Shin-Etsu, Tokyo, Japan.
11. Silica, Alumina, Titanium Dioxide, Talc with surface-coat by Dimethicone/Methicone Copolymer. Available in Catalysts & Chemicals Ind. Co. Ltd., Kawasaki, Japan.
12. 25% Dimethicone/Vinyl Dimethicone Crosspolymer in Dimethicone. Available from Shin-Etsu, Tokyo, Japan.
13. Stearyl Dimethicone wax. Available from Dow Corning, Midland, MI.
14. Polyethylene. Available from Jeen Int'l Corp., Fairfield, NJ.
15. Hydrophobically modified TiO₂ dispersion available from Kobo Products, Inc., South Plainfield, NJ.
16. Available from Procter & Gamble Chemicals, Cincinnati, OH.
17. Hexamidine Diisethionate. Availabile from Laboratoires Serobiologiques, Paris, France.
18. 0.085% Palmitoyl Pentapeptide-3 in water. Available from Sederma, Edison, NJ.
19. DMDM Hydantoin, Iodopropynyl butylcarbamate, 1,3 butylenel glycol in water. Available from Lonza Inc., Basel, Switzerland.

In separate suitable containers are added the ingredients of Phase A and Phase B and each phase is mixed using a suitable mixer (e.g., Anchor blade, propeller blade, IKA T25). When each phase is homogenous, slowly add Phase B to Phase A while mixing Phase A with a suitable mixer (e.g., Anchor blade, propeller blade, IKA T25). Maintain mixing until batch is uniform. Pour product into suitable containers.

### Test Methods

Microscopy Method

This method is a microscope-assisted visual analysis of the presence and size of the water domains within a sample composition. The method uses a standard optical microscope with Differential Interference Contrast and Crossed Polarized Light capabilities and a optical shear stage. Optionally, cross polarization may be used for sample compositions that have low translucency or for characterization of the watery domains. With the cross polarization technique, watery domains will appear dark in the resulting image. A suitable configuration includes a Zeiss Axioplan 2 microscope (available from Carl Zeiss, Inc, Thornwood, N.Y.) coupled with a MTI 3CCD camera (available from DAGE-MTI, Michigan City, Ind.). Images are acquired using Metamorph software version 6.1 (available from Molecular Devices Corporation, Sunnyvale, Calif.) that is used to measure droplet size and save the resulting image. The microscope is paired with a CSS450 optical shear stage (available from Linkam Scientific Instruments, Surrey, UK). The microscope is configured to provide 500× magnification. About 1.5 g of the emulsion (“Sample”) is carefully loaded onto the shear stage to minimize shear. The shear system is configured for a steady mode having a gap width of 1 mm and a constant shear rate of 16 s⁻¹. Temperature is held constant at approximately 25° C. An initial micrograph is captured of the Sample prior to initiation of shear by the shear stage. The sample should have an average water droplet size of about 3 microns or less. If a Sample exhibits an average water droplet size of greater than 3 microns, the Sample may not be properly characterized by microscopy; however, the Sample may be characterized by the Milling method or the Rheological method. The Sample is subjected to 15 seconds of shear, the shear is discontinued, and a micrograph is captured. This is repeated three times (e.g., Sample is subjected to a cumulative 60 seconds of shear) to yield five micrographs (e.g., taken at time=0, 15, 30, 45, and 60 seconds). The visible water domains of the Sample are analyzed to provide a maximum linear dimension for each of the visible water domains. Compositions that do not release when applied to the skin do not exhibit a significant change in the water droplet size when exposed to these conditions. Three Samples of each emulsion are tested.

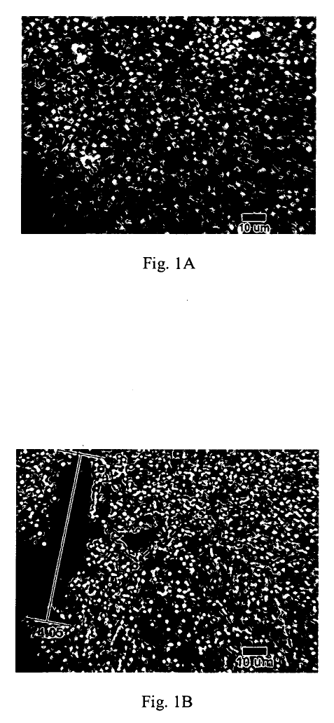

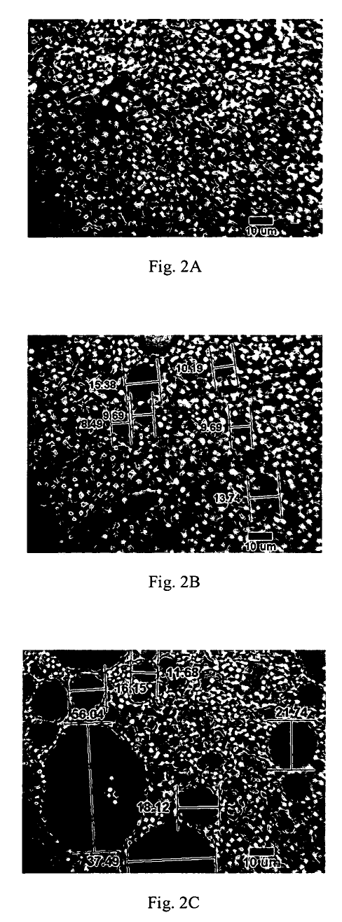

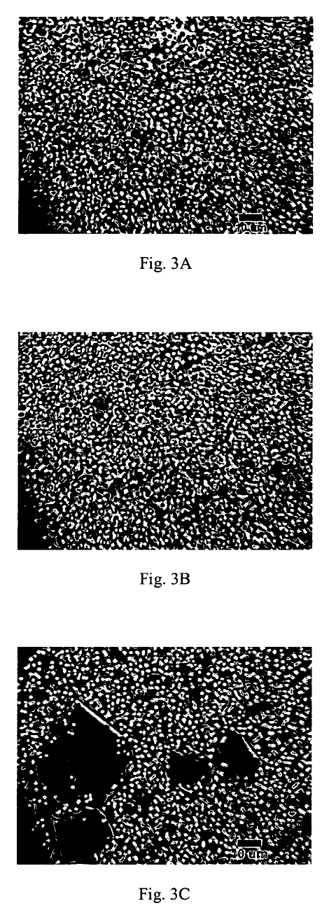

Micrographs for select examples tested according to the microscopy method are provided as FIGS. 1A-B, 2A-C, and 3A-C. The values shown in the micrographs are the approximate longest dimension (in micrometers) of the aqueous domains. FIGS. 1A-B are micrographs of Example 13 taken at 0 seconds and 15 seconds, respectively. FIG. 1B shows an aqueous domain of approximately 74.05 μm after 15 seconds of shear. FIGS. 2A-C are micrographs of Example 12 taken at 0 seconds, 15 seconds, and 60 seconds, respectively. FIG. 2C shows an aqueous domain of approximately 56.04 μm after 60 seconds of shear. FIGS. 3A-C are micrographs of a Comparative Example (commercially available Regenerist Daily Regenerating Serum available from The Procter & Gamble Company) taken at 0 seconds, 15 seconds, and 60 seconds, respectively. FIG. 3C shows silicone elastomer domains that are readily characterized to a skilled microscopist; however, no aqueous domains greater than 10 μm are present.

Milling Method

This method involves the bulk milling of the sample emulsion (“Sample”) to yield a visible (to the naked eye) phase separation. The milling method involves the bulk milling of a 30 g Sample in 50 mL beaker using an Ultra Turrax T25 mixer with a S 25 KR-18G dispersing element available from IKA Works, Wilmington, N.C. The method is conducted at a temperature of approximately 25° C. The Sample is milled for about 1 minute at a speed of either about 13500 rpm (which corresponds to a shear rate of about 30000 s⁻¹) or about 24000 rpm (which corresponds to a shear rate of about 53000 s⁻¹). During the 1 minute of milling, the beaker may be gently (i.e., reciprocating motion of no more than about 1 Hz) moved by hand in a direction parallel to the rotor axis of the mixer. Optionally, a Sample may be milled at a speed of 8000 rpm (which corresponds to a shear rate of about 18000 s⁻¹). After no more than 5 minutes after milling is ended, phase separation is visually observed. The aqueous phase is removed from the beaker using standard separation techniques. The separated aqueous phase is weighed. The method is repeated with two additional samples and the weights are averaged.

Select examples tested according to the Milling Method provide the following water release:

| | Comparative | | | |
|---|---|---|---|---|
| | Example * | Example 7 | Example 8 | Example 9 |
| 8000 rpm | 0.0 g | 2.35 g | 0.0 g | 0.0 g |
| 13500 rpm | 0.0 g | 13.68 g | 5.22 g | 0.43 g |
| 24000 rpm | 0.0 g | 18.39 g | 15.05 g | 5.68 g |

* The Comparative Example is the commercially available Regenerist Daily Regenerating Serum available from The Procter & Gamble Company.

Rheological Method

This method provides a rheological profile for the emulsion (“Sample”). The Sample is evaluated using an AR 2000 Rheometer available from TA Instruments, New Castle, Del. that is interfaced with a computer having software that provides data recordation and analysis. The rheometer is configured with 4 cm flat plates at a gap setting of 1000 microns, a temperature of 25° C., and in a controlled stress mode. The rheometer is configured to ramp stress from 1Pa to 1000 Pa with a duration of 3 minutes and to sample at a rate of 10 points per decade. A rheology profile is plotted using the log₁₀ viscosity (Pa·s) on the y-axis versus the log₁₀ shear stress (Pa) on the x-axis. Water-releasing Samples exhibit a sharp decrease in viscosity at a critical shear stress. This decrease in viscosity may be measured as the slope of the plot between the regions wherein the viscosity has a substantially constant high viscosity and a substantially constant lower viscosity. The slope is calculated according to the formula [(log viscosity(t2)−log viscosity(t1)]/[(log shear stress(t2)−log shear stress (t1)], where viscosity (t1) and viscosity (t2) are the viscosity readings before and after the viscosity value decreases 10 fold (which on the log scale is a change of 1.0) between two readings, and the shear stress (t1) and shear stress (t2) are the corresponding shear stress readings. If the viscosity decreases gradually and no sudden viscosity drop of more than 10 fold between two readings occurs, any representative readings on the plot can be used for the slope calculation. It is believed that the sharp decrease in viscosity evidences the release of water from the Sample.

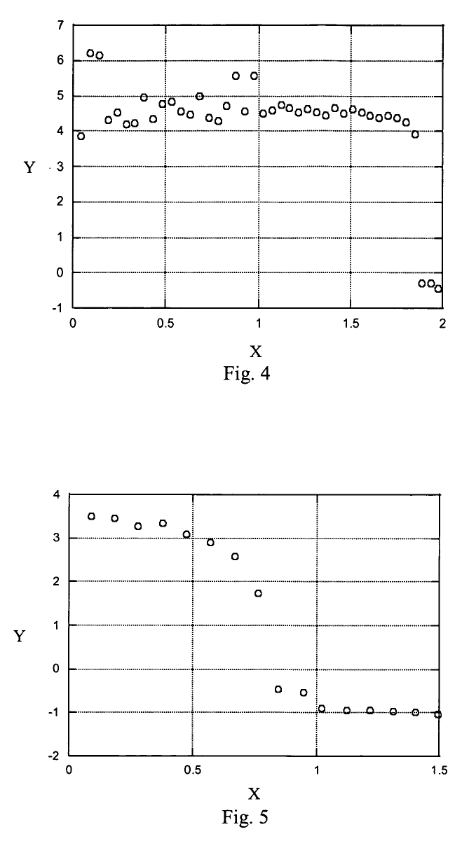

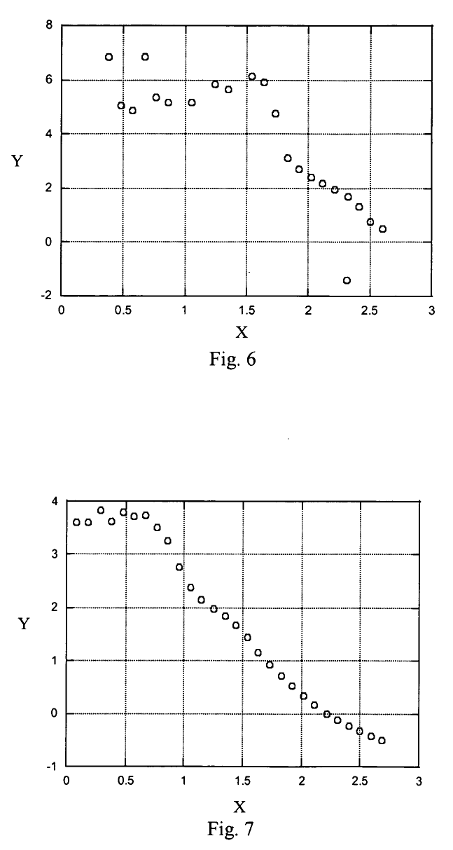

Graphs of the resulting data for select examples tested according to the rheological method are provided in FIGS. 4-7. FIG. 4 is the graph that results from Example 12. FIG. 4 shows a steep drop in viscosity (e.g., slope of about −106) between data points at a shear stress of approximately 1.8 (log). FIG. 5 is the graph that results from Example 11. FIG. 5 shows a drop in viscosity (e.g., slope of about −14.7) between data points at a shear stress of approximately 0.8 (log). FIG. 6 is the graph that results from Example 10. FIG. 6 shows a drop in viscosity (e.g., slope of about −12) between data points at a shear stress of approximately 1.7 (log). FIG. 7 is the graph that results from testing a Comparative Example (commercially available Regenerist Daily Regenerating Serum available from The Procter & Gamble Company). The largest point to point drop in viscosity for the Comparative Example is about −3.4

It is understood that the foregoing detailed description of examples and embodiments of the present invention are given merely by way of illustration, and that numerous modifications and variations may become apparent to those skilled in the art without departing from the spirit and scope of the invention; and such apparent modifications and variations are to be included in the scope of the appended claims.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A water in oil emulsion composition comprising:
a) from about 0.1% to about 15% of a non-emulsifying crosslinked siloxane elastomer;
b) from about 0.1% to about 15% of an emulsifying crosslinked siloxane elastomer;
c) from about 1% to about 40% of a solvent for the non-emulsifying and emulsifying crosslinked siloxane elastomers;
d) optionally, from 0% to about 5% of an additional emulsifier;
e) from about 50% to about 99% of aqueous phase;
wherein when shear stress is applied to the composition during spreading on skin, aqueous phase is released from the emulsion.

2. A composition according to claim 1 further comprising a skin care active selected from the group consisting of niacinamide, hexamidine compounds, whitening agents, peptides, sugar amines, and mixtures thereof.

3. A composition according to claim 2 wherein the skin care active is niacinamide.

4. A composition according to claim 2 wherein the skin care active is a whitening agent.

5. A composition according to claim 1 further comprising a skin conditioning agent.

6. A composition according to claim 5 wherein the skin conditioning agent is selected from the group consisting of propylene glycol, dipropylene glycol, polypropylene glycol, polyethylene glycol, sorbitol, hydroxypropyl sorbitol, trehalose, hexylene glycol, 1,3-butylene glycol, 1,2,6-hexanetriol, glycerin, 1,2-hexanediol, pentylene glycol, ethoxylated glycerin, propoxylated glycerin, and mixtures thereof.

7. A composition according to claim 6 wherein the skin conditioning agent is glycerin.

8. A composition according to claim 1 further comprising a particulate material.

9. A composition according to claim 1, wherein the emulsifying crosslinked siloxane elastomer is dimethicone copolyol crosspolymer and dimethicone.

10. A composition according to claim 1, wherein the non-emulsifying crosslinked siloxane elastomer is selected from the group consisting of dimethicone/vinyl dimethicone crosspolymers, and mixtures thereof.

12. A composition according to claim 1, wherein the solvent for the elastomer is selected from the group consisting of volatile, non-polar oils; non-volatile, polar oils; non-volatile, non-polar oils; non-volatile paraffinic hydrocarbon oils; and mixtures thereof.

13. A composition according to claim 1, wherein the composition further comprises from about 0.1% to about 50% of an additional skin care active selected from the group consisting of sunscreen actives, oil-soluble terpene alcohols, phytosterol, oil-soluble vitamin compounds, emollients and occlusives, dehydroacetic acid, hexanediol, anti-acne actives, beta hydroxy acids, chelators, flavanoids, anti-inflammatory agents, anti-cellulite agents, topical anesthetics, desquamation actives, anti-oxidants/free radical scavengers, tanning actives, skin soothing and healing agents, anti-microbial actives, anti-fungal actives, and mixtures thereof.

14. A method of regulating the condition of skin, said method comprising applying to the skin of a human in need of treatment, a safe and effective amount of a composition according to claim 1.

15. A water in oil emulsion composition comprising:
a) from about 0.1% to about 15% of a non-emulsifying crosslinked siloxane elastomer;
b) from about 0.1% to about 15% of an emulsifying crosslinked siloxane elastomer;
c) from about 1% to about 40% of a solvent for the non-emulsifying and emulsifying crosslinked siloxane elastomers;
d) optionally, from 0% to about 5% of an additional emulsifier;
e) from about 50% to about 99% of aqueous phase;
wherein said composition releases of an amorphous region of the aqueous phase having a maximum linear dimension of at least about 10 microns within about 1 minute of shear according to Microscopy Method described herein.

16. A water in oil emulsion composition comprising:
a) from about 0.1% to about 15% of a non-emulsifying crosslinked siloxane elastomer;
b) from about 0.1% to about 15% of an emulsifying crosslinked siloxane elastomer;
c) from about 1% to about 40% of a solvent for the non-emulsifying and emulsifying crosslinked siloxane elastomers;
d) optionally, from 0% to about 5% of an additional emulsifier;
e) from about 50% to about 99% of aqueous phase;
wherein said composition releases a portion of the aqueous phase weighing at least 0.25 g after 1 minute of milling at a rate of 13500 rpm according to Milling Method described herein.

17. A water in oil emulsion composition comprising:
a) from about 0.1% to about 15% of a non-emulsifying crosslinked siloxane elastomer;
b) from about 0.1% to about 15% of an emulsifying crosslinked siloxane elastomer;
c) from about 1% to about 40% of a solvent for the non-emulsifying and emulsifying crosslinked siloxane elastomers;
d) optionally, from 0% to about 5% of an additional emulsifier;
e) from about 50% to about 99% of aqueous phase;
wherein said composition yields a plot of log shear stress versus log viscosity having a slope of less than −5 as measured according to the Rheological Method described herein.
